(19) United States
(12) Patent Application Publication
(10) Pub. No.: US 2025/0388612 A1
(43) Pub. Date: Dec. 25, 2025

(54) METAL IRIDIUM COMPLEX AND APPLICATION THEREOF

(71) Applicant: SICHUAN AG-RAY NEW MATERIALS CO., LTD, Meishan City, Sichuan (CN)

(72) Inventors: Liangliang YAN, Guangdong (CN); Lei DAI, Guangdong (CN); Lifei CAI, Guangdong (CN)

(73) Assignee: SICHUAN AG-RAY NEW MATERIALS CO., LTD, Meishan City, Sichuan (CN)

(21) Appl. No.: 18/693,655

(22) PCT Filed: Aug. 30, 2022
(86) PCT No.: PCT/CN2022/115660

§ 371 Date: Apr. 22, 2025

(30) Foreign Application Priority Data
Sep. 28, 2021 (CN) 202111142945.3

(51) Int. Cl. C07F 15/00; C09K 11/06; H10K 50/12; H10K 50/17; H10K 85/30

(52) U.S. Cl. CPC C07F 15/0033; C09K 11/06; H10K 50/12; H10K 50/17; H10K 85/342; C09K 2211/185

(57) ABSTRACT

The present invention relates to an organometallic iridium compound and application thereof. The organometallic iridium compound has a general formula of Ir(La)(Lb)(Lc), where La is a structure as shown in a formula (1), and Lb is a structure as shown in a formula (2). The compound provided in the present invention has the advantages of low sublimation temperature, good optical and electrical stability, high luminous efficiency, long service life, high color saturation and the like, and can be used in an organic light-emitting device. In particular, the compound has the possibility of being applied to the AMOLED industry as a red light-emitting phosphorescent material, especially for display, illumination and vehicle taillights.

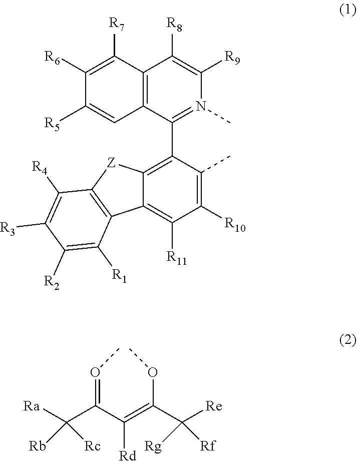

### TECHNICAL FIELD

The present invention relates to the technical field of organic electroluminescence, in particular to an organic light-emitting material, and specially relates to a metallic iridium compound and application thereof in an organic electroluminescent device.

### BACKGROUND

At present, as a new-generation display technology, an organic electroluminescent device (OLED) has attracted more and more attention in display and lighting technologies, thus having a wide application prospect. However, compared with market application requirements, properties, such as luminous efficiency, driving voltage and service life, of the OLED still need to be strengthened and improved.

In generally, the OLED includes various organic functional material films with different functions sandwiched between metal electrodes as a basic structure, which is similar to a sandwich structure. Under the driving of a current, holes and electrons are injected from a cathode and an anode, respectively. After moving to a certain distance, the holes and the electrons are compounded in a light-emitting layer, and then released in the form of light or heat to achieve luminescence of the OLED.

However, organic functional materials are core components of the OLED, and the thermal stability, photochemical stability, electrochemical stability, quantum yield, film forming stability, crystallinity, color saturation and the like of the materials are main factors affecting properties of the device.

Generally, the organic functional materials include fluorescent materials and phosphorescent materials. The fluorescent materials are usually organic small-molecule materials, which can only utilize 25% of a singlet state for luminescence, so that the luminous efficiency is relatively low. Meanwhile, due to an earth-spin orbit coupling effect caused by a heavy atom effect, the phosphorescent materials can utilize 25% of a singlet state and can also utilize 75% of the energy of triplet excitons, so that the luminous efficiency can be improved. However, compared with the fluorescent materials, the phosphorescent materials are developed later, and the thermal stability, service life, color saturation and the like of the materials need to be improved. Thus, the phosphorescent materials have become a challenging topic. Various compounds have been developed to serve as the phosphorescent materials. For example, an invention patent document CN107973823 discloses a quinoline iridium compound, but the color saturation and device properties, especially luminous efficiency and device service life, of the compound need to be improved. An invention patent document CN106459114 discloses an iridium compound coordinated with a β-dione coordination group, but the compound has high sublimation temperature, poor color saturation, and particularly unsatisfactory device properties, especially luminous efficiency and device service life, which need to be further improved. In addition, a patent document CN111377969 discloses an iridium compound of dibenzofuran-isoquinoline

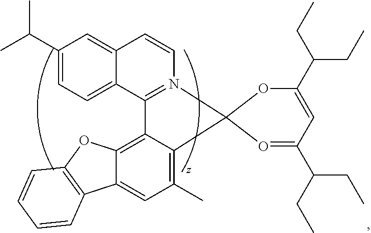

but device properties, especially the color saturation, of the two materials cannot meet demands of a display color gamut of BT2020, which need to be further improved so as to meet demands of a rapidly developing market for OLED light-emitting materials.

### SUMMARY

In order to solve the above problems, the present invention provides an organic electroluminescent device having high properties and a novel material capable of realizing the organic electroluminescent device.

In order to achieve the above purposes, the inventor has conducted in-depth studies repeatedly and found that an organic electroluminescent device having high properties can be obtained by using an organometallic iridium compound containing structures as shown in a formula (1) and a formula (2) as ligands.

The organometallic iridium compound has a general formula of Ir(La)(Lb)(Lc), where La is a structure as shown in a formula (1), and Lb is a structure as shown in a formula (2). The compound provided in the present invention has the advantages of low sublimation temperature, good optical and electrical stability, high luminous efficiency, long service life, high color saturation and the like, and can be used in an organic light-emitting device. In particular, the compound has the possibility of being applied to the AMOLED industry as a red light-emitting phosphorescent material, especially for display, illumination and vehicle taillights.

An organometallic iridium compound has a general formula of Ir(La)(Lb)(Lc), where La is a structure as shown in a formula (1):

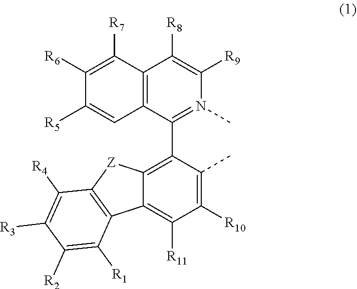

where a dotted line refers to a position connected to the metal Ir;
Z is O, S or Se;
R₁-R₁₁ are independently selected from hydrogen, deuterium, halogen, cyano, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₃-C₂₀heterocycloalkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl, or two adjacent groups of R₁-R₄ are connected to each other to form an aliphatic ring;
R₁₀ is not hydrogen, deuterium, halogen or cyano;
at least one of R₅-R₇ is substituted or unsubstituted C₆-C₃₀ aryl, or substituted or unsubstituted C₂-C₃₀ heteroaryl;
the heteroalkyl, the heterocycloalkyl and the heteroaryl at least contain one O, N, or S heteroatom;
and the “substituted” refers to substitution with deuterium, F, Cl, Br, C₁-C₆ alkyl, C₃-C₆ cycloalkyl, C₁-C₆ alkyl substituted amino, cyano, isocyano, or phosphino, and the substitution ranges from a single substitution number to a maximum substitution number;
Lb is a structure as shown in a formula (2):

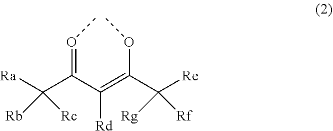

where a dotted line refers to a position connected to the metal Ir;
Ra-Rg are independently selected from hydrogen, deuterium, halogen, substituted or unsubstituted C₁-C₁₀ alkyl, substituted or unsubstituted C₃-C₂₀ cycloalkyl, substituted or unsubstituted C₁-C₁₀heteroalkyl, and substituted or unsubstituted C₃-C₂₀heterocycloalkyl, or any two of Ra, Rb and Rc are connected to form an aliphatic ring, and any two of Re, Rf and Rg are connected to form an aliphatic ring;
the heteroalkyl and the heterocycloalkyl at least contain one O, N, or S heteroatom;
and the “substituted” refers to substitution with deuterium, F, Cl, Br, C₁-C₄ alkyl, C₁-C₄ alkoxyl, C₃-C₆ cycloalkyl, C₁-C₄
alkyl substituted amino, cyano,, isocyano, or phosphino;
Lc is a monoanionic bidentate ligand, and the Lc is different from the Lb and is not an OO ligand;
the Lc and the La are the same or different, and the different indicates different parent nuclear structures, a same parent nuclear structure with different substituents, or a same parent nuclear structure with same substituents at different substituent positions;
and any two or three of the La, the Lb and the Lc are connected to each other to form a polydentate ligand.

As a optional organometallic iridium compound, the R₆ is substituted or unsubstituted C₆-C₃₀ alkyl, or substituted or unsubstituted C₂-C₃₀ heteroaryl.

As a optional organometallic iridium compound, the R₆ is substituted or unsubstituted C₆-C₁₈ alkyl, or substituted or unsubstituted C₂-C₁₇ heteroaryl.

As a optional organometallic iridium compound, the R₁₀ is optionally substituted or unsubstituted C₁-C₆ alkyl, or substituted or unsubstituted C₃-C₆cycloalkyl, and the “substituted” refers to substitution with deuterium, F, C₁-C₅ alkyl, or C₃-C₆ cycloalkyl.

As a optional organometallic iridium compound, at least one of the R₈ and the R₉ is not hydrogen, deuterium, halogen or cyano.

As a optional organometallic iridium compound, at least one of the R₈ and the R₉ is substituted or unsubstituted C₁-C₆ alkyl, or substituted or unsubstituted C₃-C₆ cycloalkyl.

As a optional organometallic iridium compound, the R₁-R₄ are hydrogen.

As a optional organometallic iridium compound, the Z is O.

As a optional organometallic iridium compound, the Lc and the La are different.

As a optional organometallic iridium compound, the Lc is a structure as shown in a formula (3):

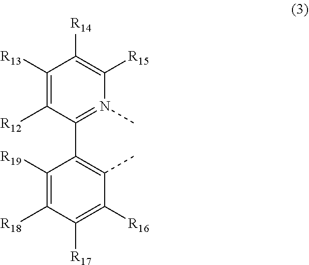

where a dotted line refers to a position connected to the metal Ir;
R₁₂-R₁₉ are independently selected from hydrogen, deuterium, halogen, cyano, hydroxyl, amino, amino group, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₂-C₁₀ alkenyl, substituted or unsubstituted C₂-C₁₀ alkynyl, substituted or unsubstituted C₆-C₁₈ aryl, substituted or unsubstituted C₂-C₁₇ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl;
at least two of the R₁₆-R₁₉ are not hydrogen;
and at least one group pf two adjacent groups of the R₁₂-R₁₅ may form an aromatic ring as shown in a formula (4) below:

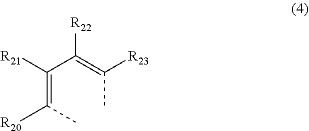

where in the formula (4),
a dotted line refers to a position connected to a pyridine ring;
R₂₀-R₂₃ are independently selected from hydrogen, deuterium, halogen, cyano, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₂-C₁₀ alkenyl, substituted or unsubstituted C₂-C₁₀ alkynyl, substituted or unsubstituted C₆-C₁₈ aryl, substituted or unsubstituted C₂-C₁₇ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl, or two adjacent groups of the R₂₀-R₂₃ are connected to each other to form an aliphatic ring or an aromatic ring;
the heteroalkyl and the heteroaryl at least contain one O, N, or S heteroatom;
and the “substituted” refers to substitution with deuterium, F, Cl, Br, C₁-C₆ alkyl, C₃-C₆cycloalkyl, C₁-C₆alkyl substituted amino, cyano, isocyano, or phosphino, and the substitution ranges from a single substitution number to a maximum substitution number.

As a optional organometallic iridium compound, the La has one of the following structural formulas, or is partially or completely deuterated or fluorinated correspondingly,

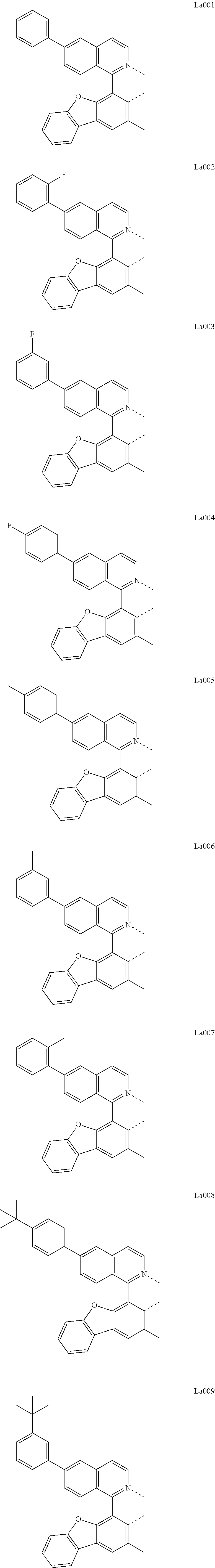

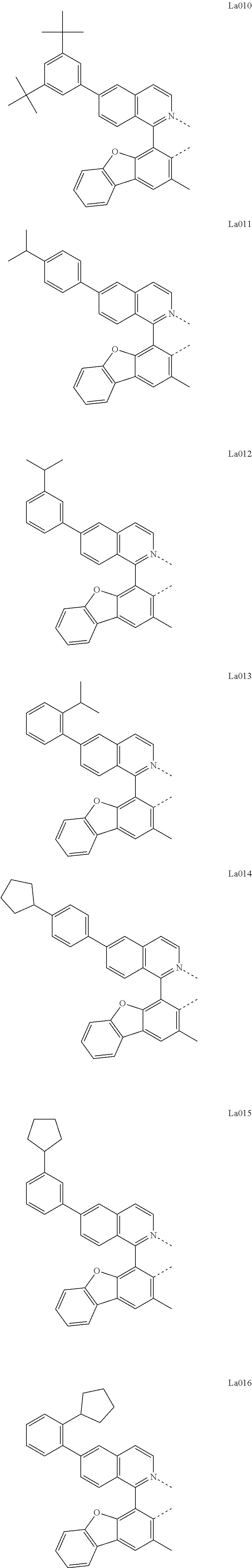

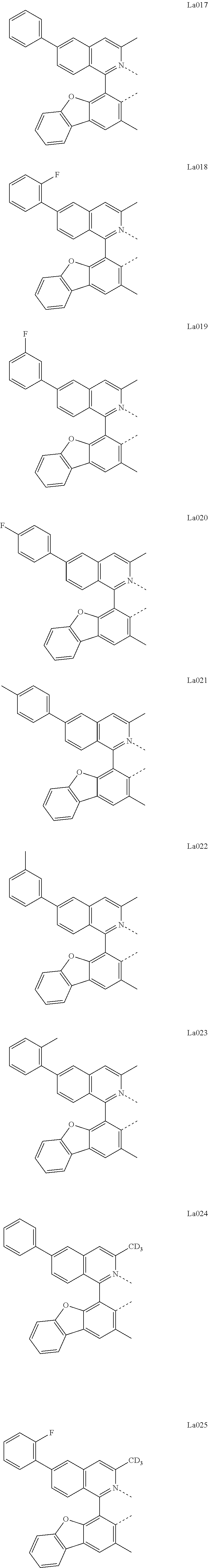

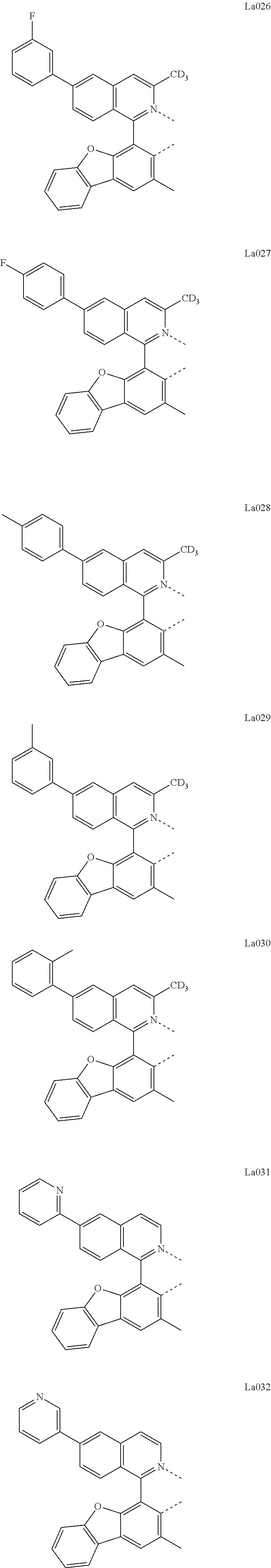

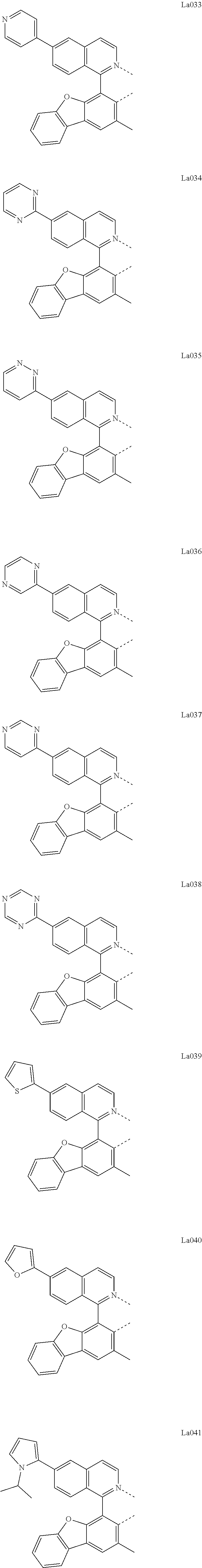

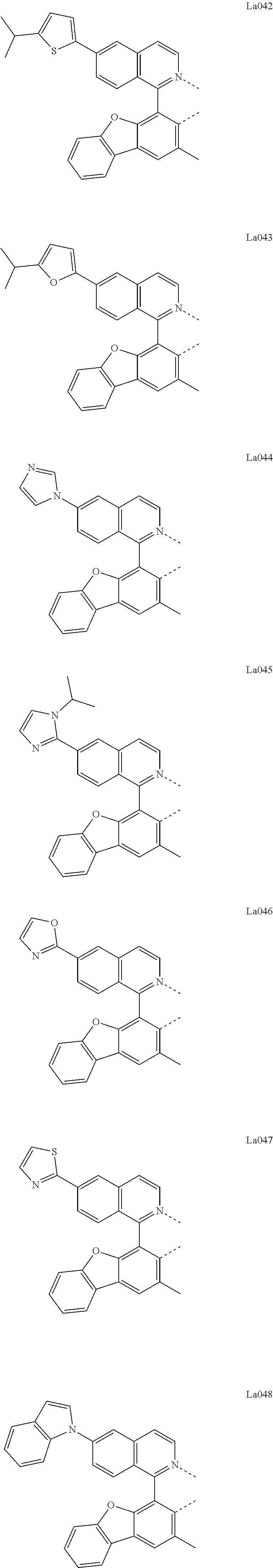

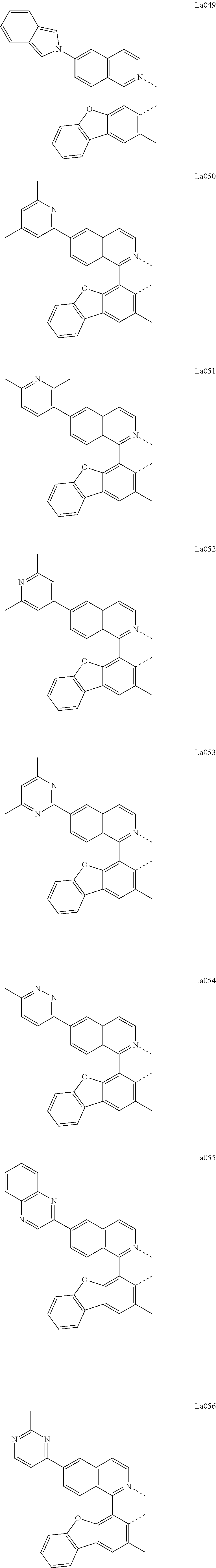

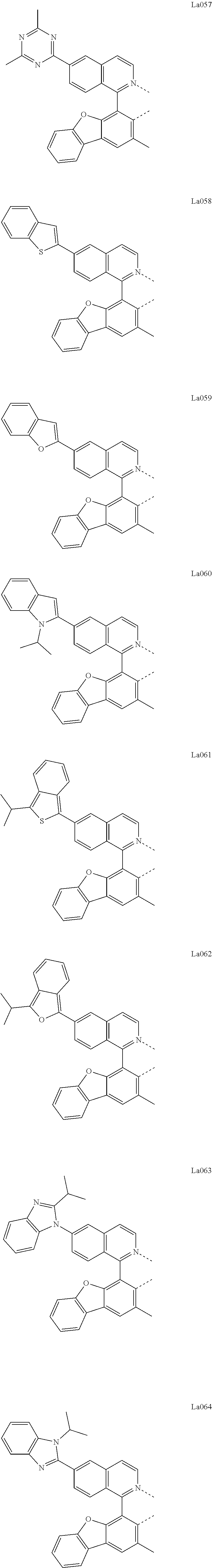

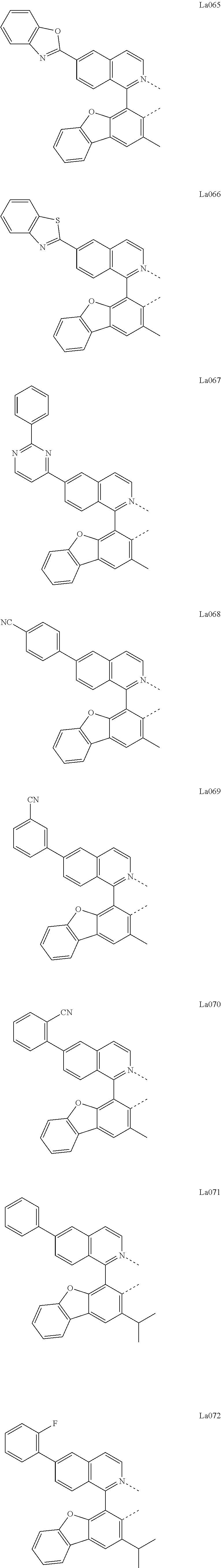

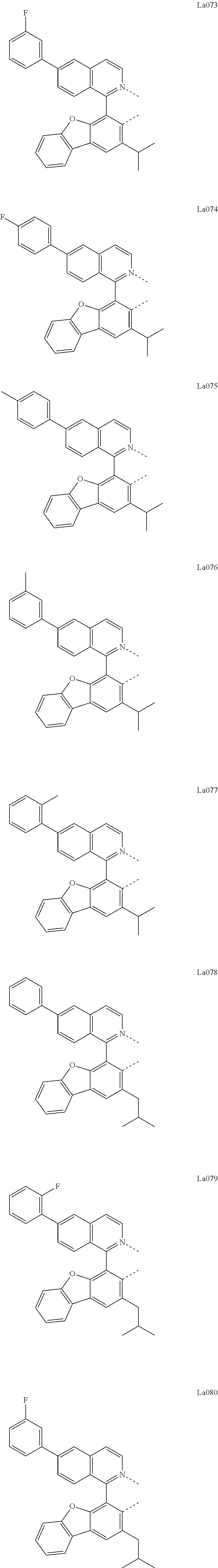

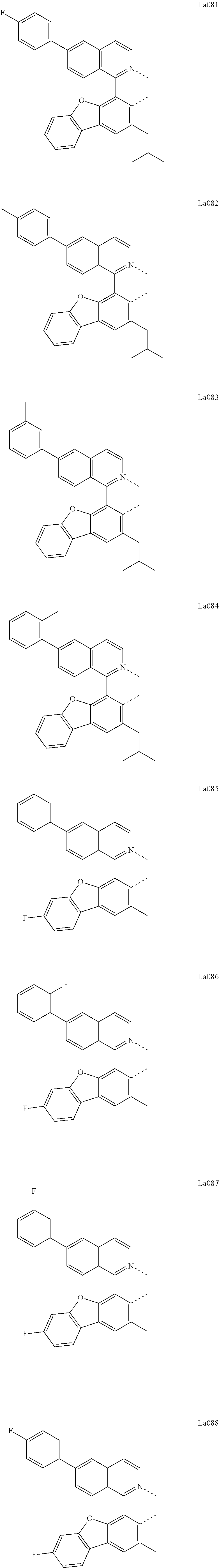

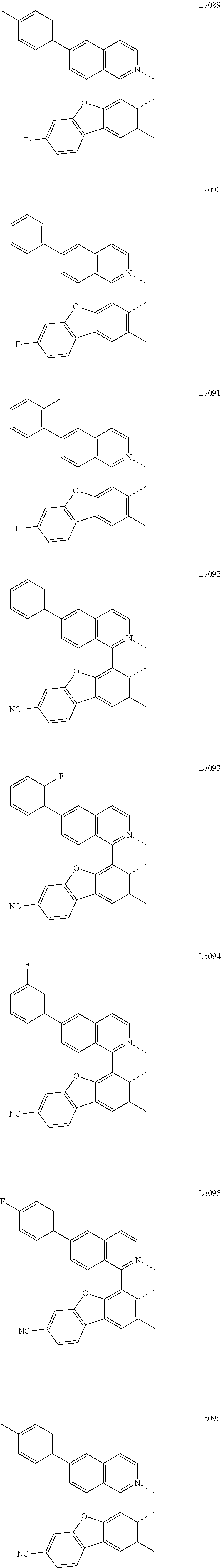

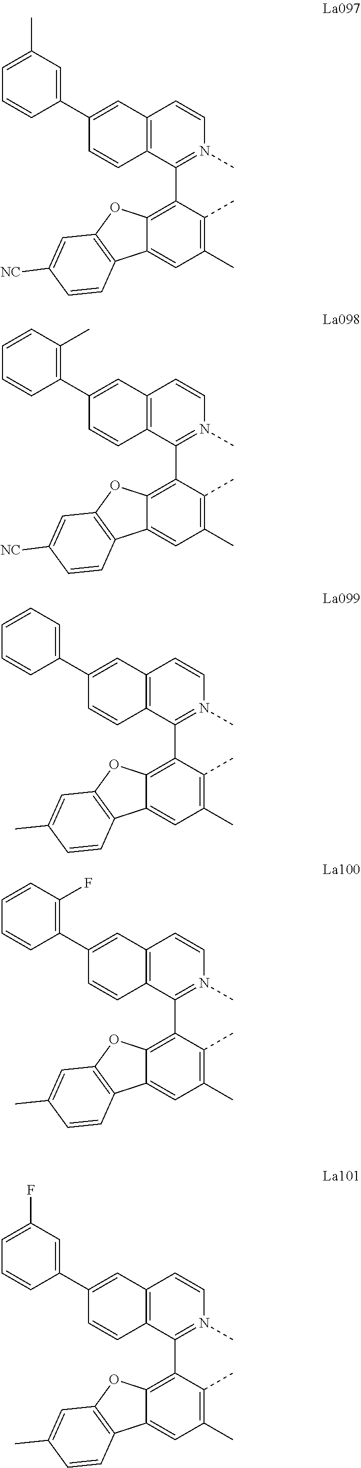

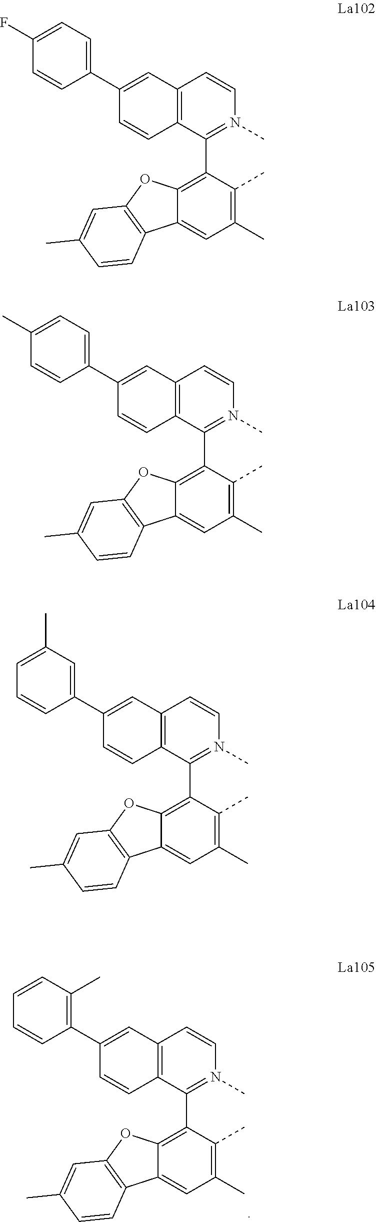

As a optional organometallic iridium compound. the Lb has one of the following structural formulas, or is partially or completely deutcrated or fluorinated correspondingly,

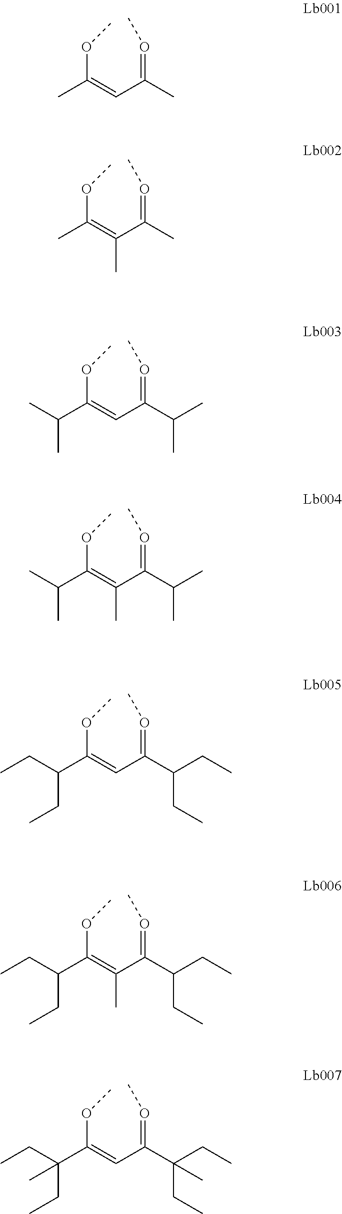

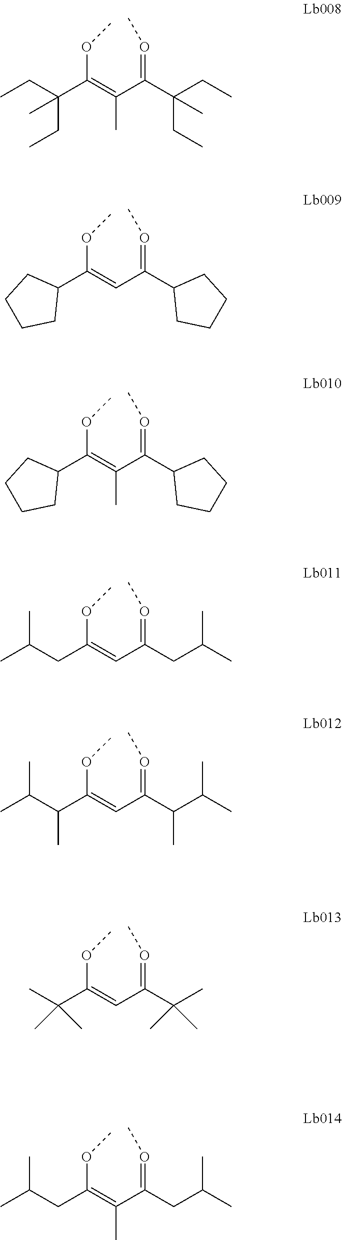

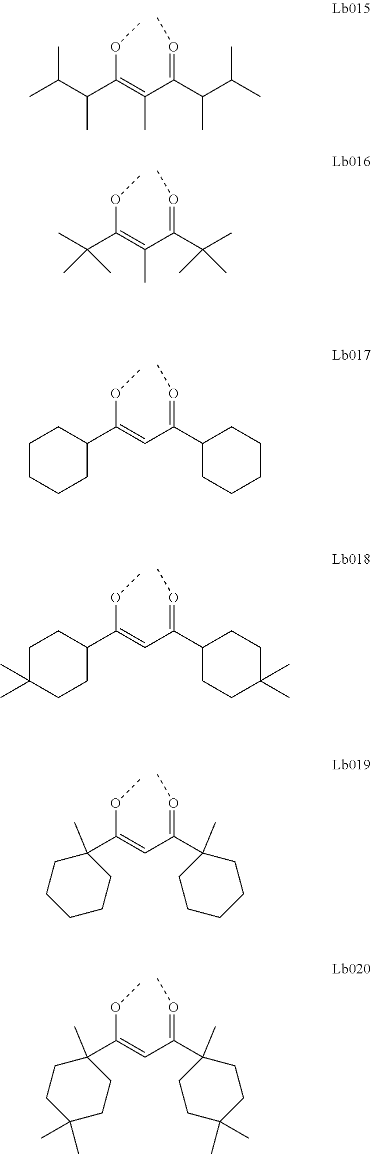

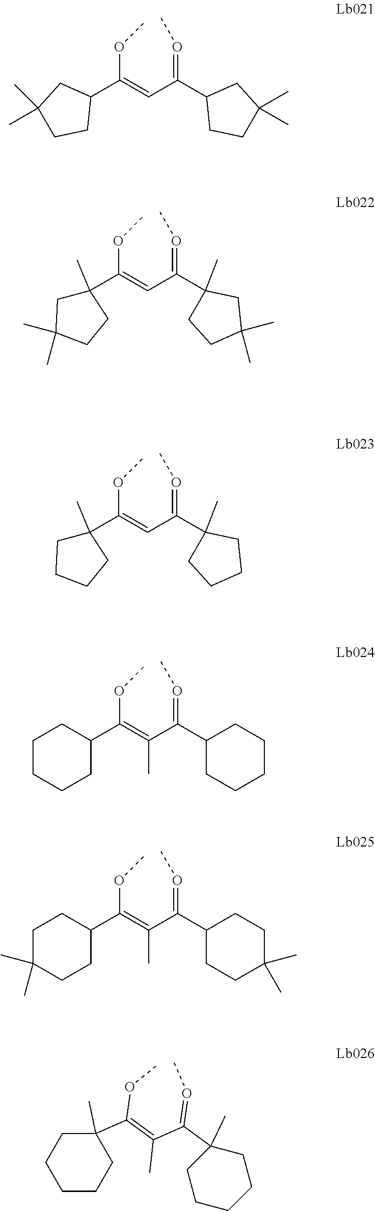

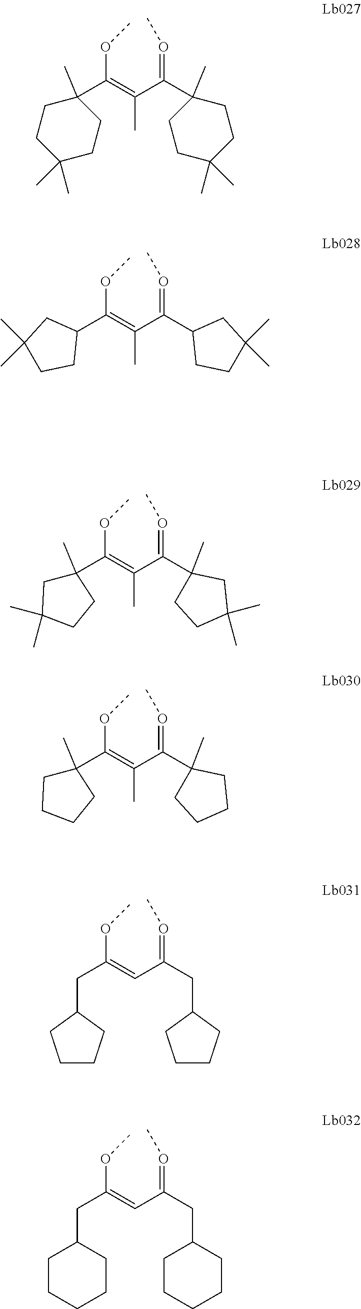

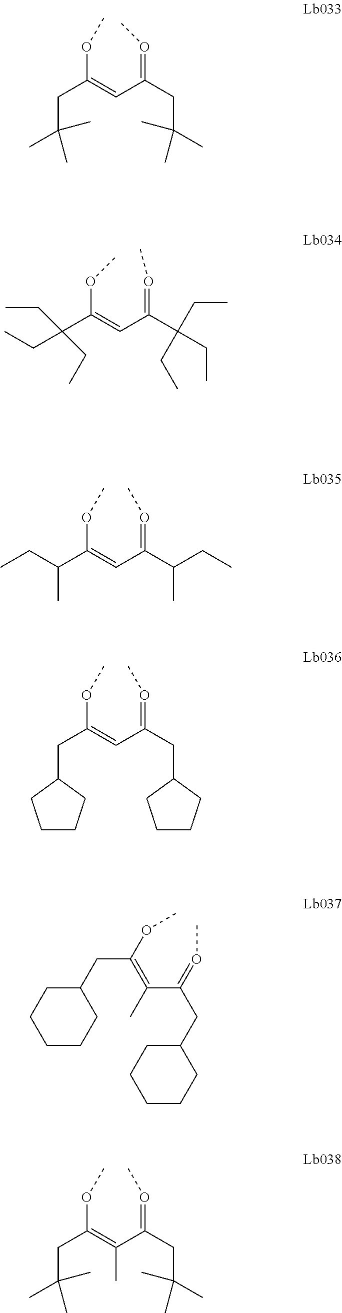

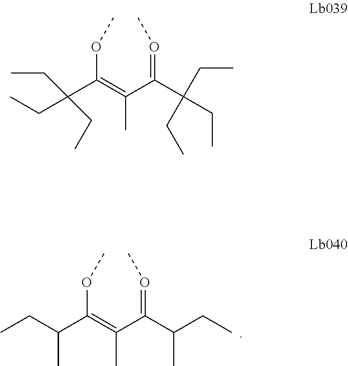

As a optional organometallic iridium compound. the Lc has one of the following structural formulas, or is partially or completely deuterated or fluorinated correspondingly,

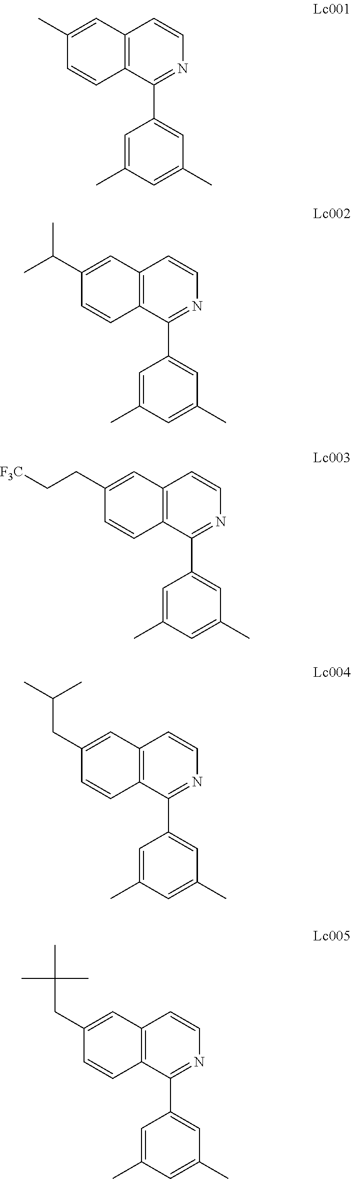

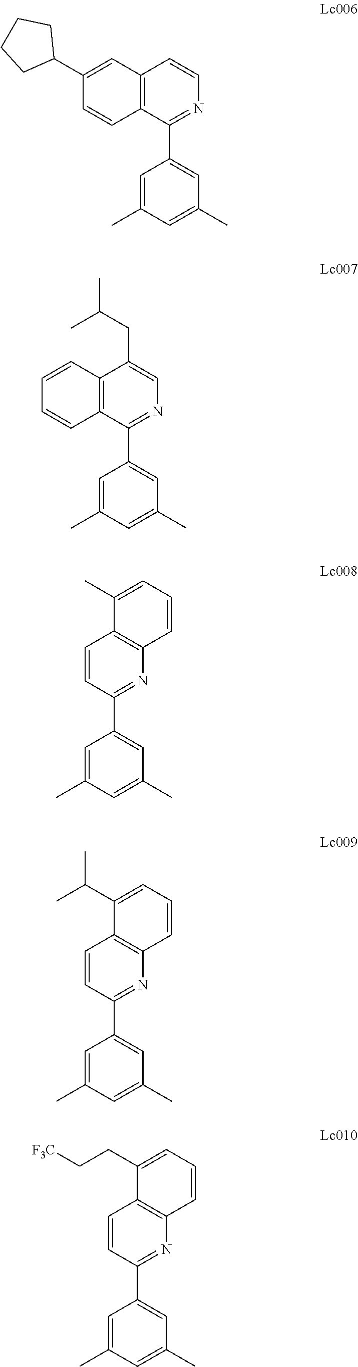

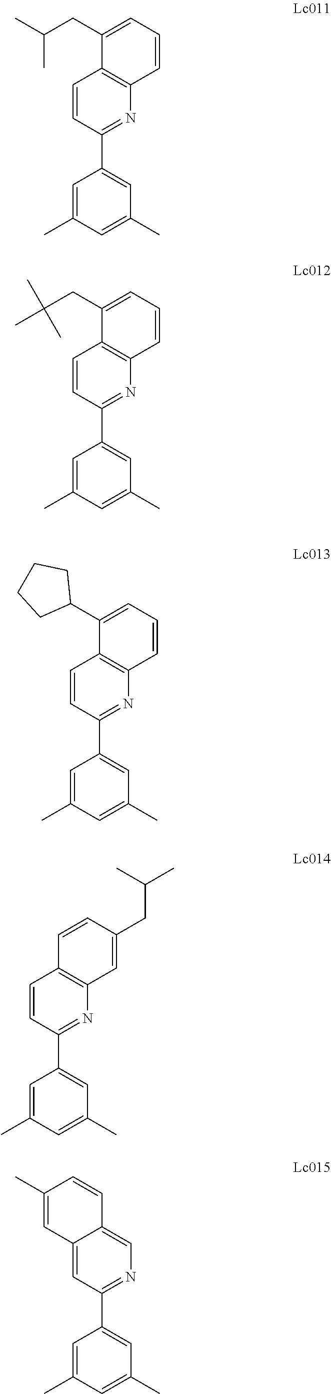

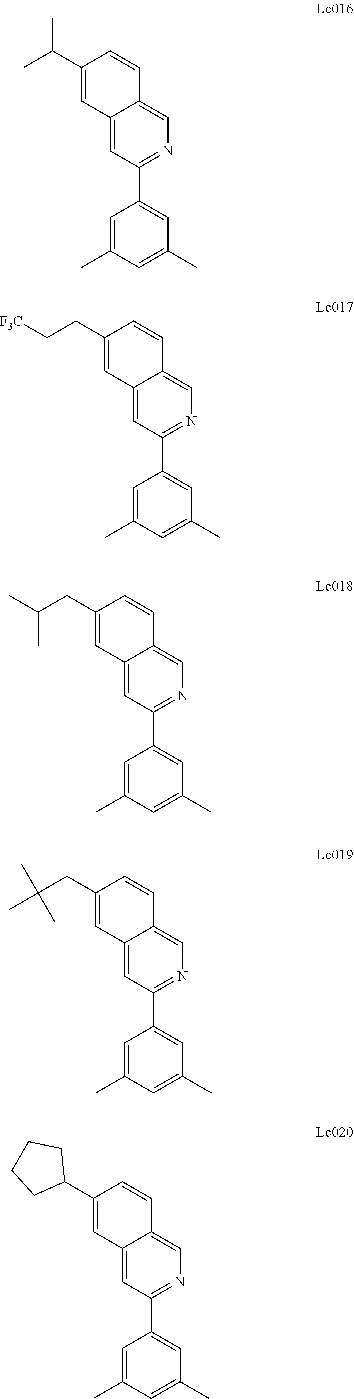

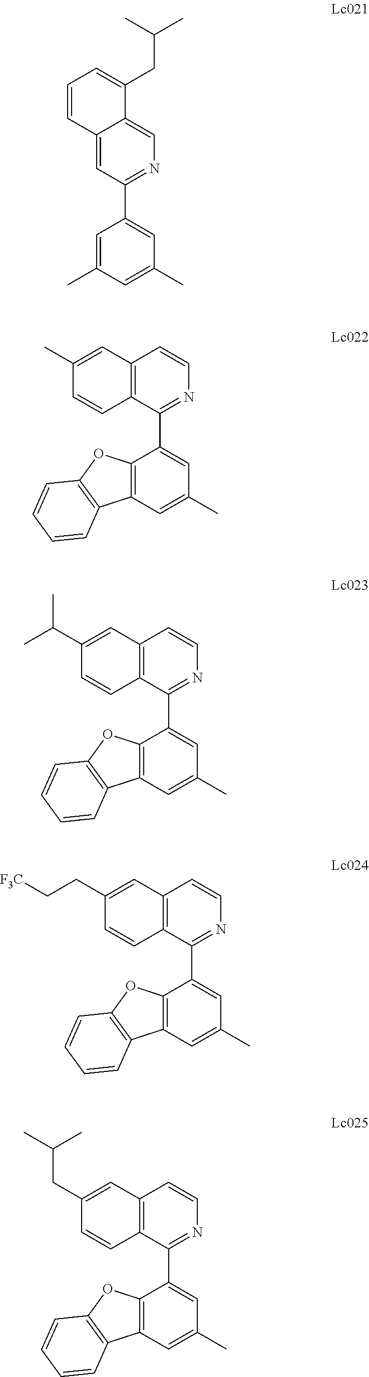

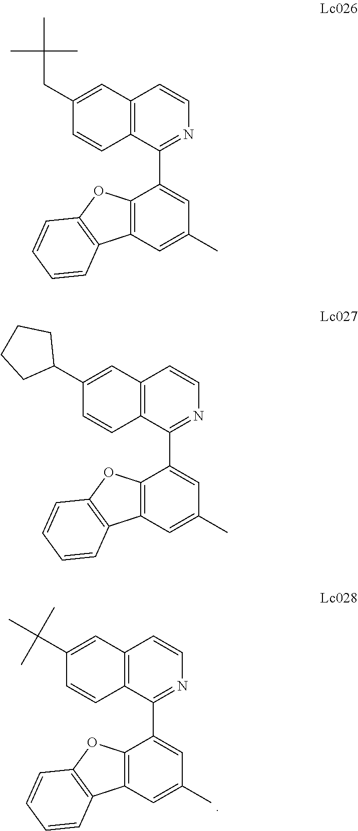

A ligand La has a structural formula below:

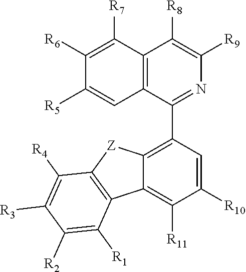

where R₁-R₁₁ and Z are defined as above.

On of purposes of the present invention is to provide an electroluminescent device. The electroluminescent device includes a cathode, an anode and an organic layer arranged between the cathode and the anode, and the organic layer includes the organometallic iridium compound.

The organic layer includes a light-emitting layer, and the metallic iridium compound is used a red light-emitting doping material for the light-emitting layer; or the organic layer includes a hole injection layer, and the metallic iridium compound is used as a hole injection material in the hole injection layer.

The material of the present invention not only has the advantages of low sublimation temperature, high optical and electrochemical stability, high color saturation, high luminous efficiency, long device service life and the like, but also can be used in an organic light-emitting device. In particular, the compound has the possibility of being applied to the AMOLED industry as a red light-emitting phosphorescent material, especially for display, illumination and vehicle taillights. The material of the present invention can convert a triplet state into light, thereby improving the luminous efficiency of organic electroluminescent devices and reducing energy consumption.

### BRIEF DESCRIPTION OF DRAWINGS

FIG. 1 shows a ¹HNMR spectrum of a compound La002 of the present invention in a deuterated chloroform solution.

FIG. 2 shows a ¹HNMR spectrum of a compound Ir(La002)₂Lb005 of the present invention in a deuterated chloroform solution.

FIG. 3 shows a ¹HNMR spectrum of a compound La005 of the present invention in a deuterated chloroform solution.

FIG. 4 shows a ¹HNMR spectrum of a compound Ir(La005)₂Lb005 of the present invention in a deuterated chloroform solution.

FIG. 5 shows an ultraviolet absorption spectrum and an emission spectrum of the compound Ir(La002)₂Lb005 of the present invention in a dichloromethane solution.

FIG. 6 shows an ultraviolet absorption spectrum and an emission spectrum of the compound Ir(La005)₂Lb005 of the present invention in a dichloromethane solution.

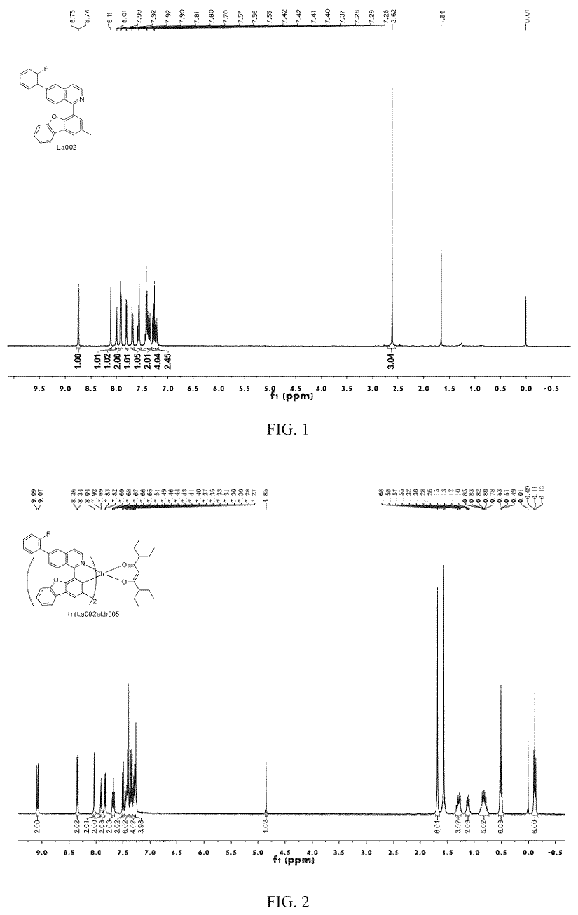

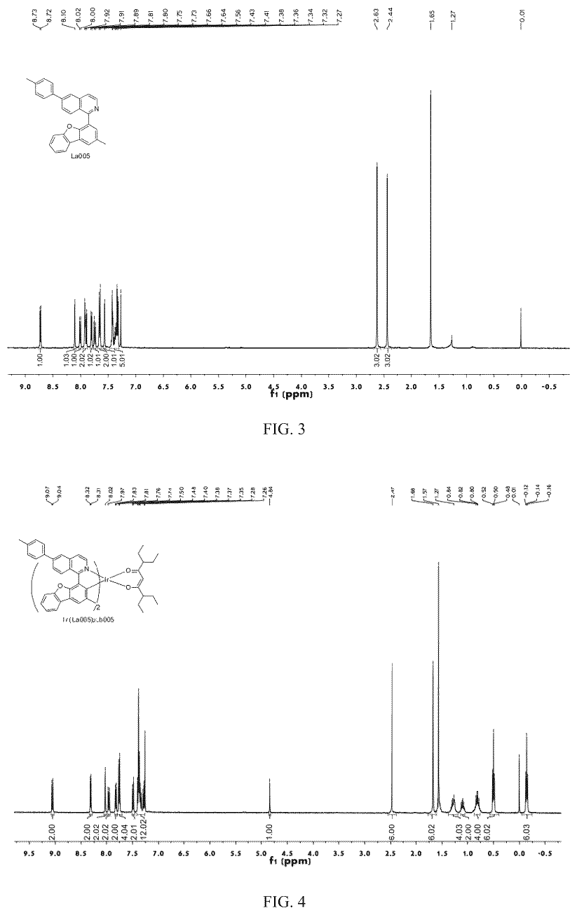

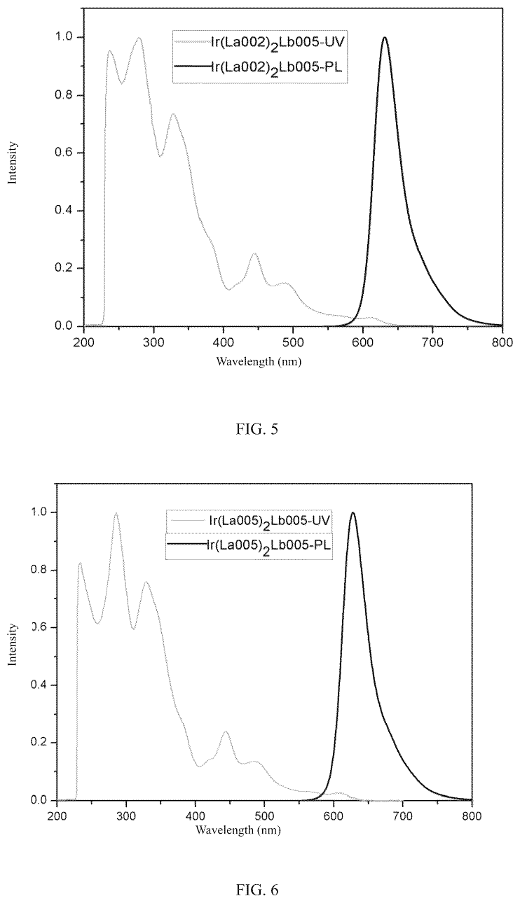

### DETAILED DESCRIPTION OF EMBODIMENTS

An organometallic iridium compound of the present invention has a general formula of Ir(La)(Lb)(Lc), where La is a structure as shown in a formula (1):

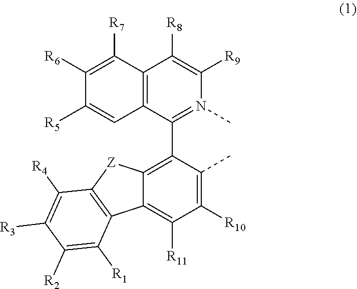

where a dotted line refers to a position connected to the metal Ir;
Z is O, S or Se;
R₁-R₁₁ are independently selected from hydrogen, deuterium, halogen, cyano, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₃-C₂₀heterocycloalkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl, or two adjacent groups of R₁-R₄ are connected to each other to form an aliphatic ring;
R₁₀ is not hydrogen, deuterium, halogen or cyano;
at least one of R₅-R₇ is substituted or unsubstituted C₆-C₃₀ aryl, or substituted or unsubstituted C₂-C₃₀ heteroaryl;
the heteroalkyl, the heterocycloalkyl and the heteroaryl at least contain one O, N, or S heteroatom;
and the “substituted” refers to substitution with deuterium, F, Cl, Br, C₁-C₆ alkyl, C₃-C₆ cycloalkyl, C₁-C₆ alkyl substituted amino, cyano, isocyano, or phosphino, and the substitution ranges from a single substitution number to a maximum substitution number;
Lb is a structure as shown in a formula (2):

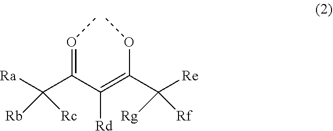

where a dotted line refers to a position connected to the metal Ir;
Ra-Rg are independently selected from hydrogen, deuterium, halogen, substituted or unsubstituted C₁-C₁₀ alkyl, substituted or unsubstituted C₃-C₂₀ cycloalkyl, substituted or unsubstituted C₁-C₁₀heteroalkyl, and substituted or unsubstituted C₃-C₂₀heterocycloalkyl, or any two of Ra, Rb and Rc are connected to form an aliphatic ring, and any two of Re, Rf and Rg are connected to form an aliphatic ring;
the heteroalkyl and the heterocycloalkyl at least contain one O, N, or S heteroatom;
and the “substituted” refers to substitution with deuterium, F, Cl, Br, C₁-C₄ alkyl, C₁-C₄ alkoxyl, C₃-C₆cycloalkyl, C₁-C₄ alkyl substituted amino, cyano, isocyano, or phosphino;
Lc is a monoanionic bidentate ligand, and the Lc is different from the Lb and is not an OO ligand;
the Lc and the La are the same or different, and the different indicates different parent nuclear structures, a same parent nuclear structure with different substituents, or a same parent nuclear structure with same substituents at different substituent positions;
and any two or three of the La, the Lb and the Lc are connected to each other to form a polydentate ligand.

Examples of various groups of componnds represented by the formula (1) to the formula (4) are described below.

It is to be noted that in the specification, “Cₐ-C_{b}” in the term “substituted or unsubstituted Cₐ-C_{b} X group” refers to the number of carbons when the X group is unsubstituted, excluding the number of carbons of a substituent when the X group is substituted.

As a linear or branched alkyl, the C₁-C₁₀ alkyl specifically includes methyl, ethyl, propyl, isopropyl, n-butyl, isobutyl, sec-butyl, tert-butyl, n-pentyl and isomers thereof, n-hexyl and isomers thereof, n-heptyl and isomers thereof, n-octyl and isomers thereof, n-nonyl and isomers thereof, and n-decyl and isomers thereof, optionally includes methyl, ethyl, propyl, isopropyl, n-butyl, isobutyl, sec-butyl, and tert-butyl, and more optionally includes propyl, isopropyl, isobutyl, sec-butyl, and tert-butyl.

The C₃-C₂₀ cycloalkyl may include cyclopropyl, cyclobutyl, cyclopentyl, cyclohexyl, 1-adamantyl, 2-adamantyl, 1-norbornyl, and 2-norbornyl, and optionally includes cyclopentyl and cyclohexyl.

The C₃-C₁₀heterocycloalkyl may include oxacyclopropyl, thiocyclobutyl, azacyclopentyl, oxacyclopentyl, oxacyclohexyl, dioxacyclohexyl and the like, optionally oxacyclopentyl and oxacyclohexyl.

The C₂-C₁₀ alkenyl may include vinyl, propenyl, allyl, 1-butadienyl, 2-butadienyl, 1-hexatrienyl, 2-hexatrienyl, and 3-hexatrienyl, and optionally includes propenyl and allyl.

As a linear or branched alkyl or cycloalkyl consisting of atoms other than carbon and hydrogen, the C₁-C₁₀heteroalkyl may include mercaptomethyl methyl, methoxymethyl, ethoxymethyl, tert-butoxyl methyl, N,N-dimethyl methyl, epoxy butyl, epoxy pentyl, and epoxy hexyl, and optionally includes methoxymethyl and epoxy pentyl.

Specific examples of the aryl include phenyl, naphthyl, anthracyl, phenanthryl, tetracenyl, pyrenyl, chrysenyl, benzo[c]phenanthryl, benzo[g]chrysenyl, fluorenyl, benzofluorenyl, dibenzofluorenyl, biphenyl, triphenyl, tetraphenyl, and fluoranthracyl, optionally phenyl and naphthyl.

Specific examples of the heteroaryl may include pyrrolyl, pyrazinyl, pyridyl, pyrimidinyl, triazinyl, indolyl, isoindolyl, imidazolyl, furyl, benzofuryl, isobenzofuryl, dibenzofuryl, dibenzothienyl, azodibenzofuryl, azodibenzothienyl, diazodibenzofuryl, diazodibenzothienyl, quinolyl, isoquinolyl, quinoxalinyl, carbazolyl, phenanthridinyl, acridinyl, phenanthrolinyl, phenazinyl, phenothiazinyl, phenoxazinyl, oxazolinyl, oxadiazolyl, furzanyl, thienyl, benzothienyl, dihydroacridinyl, azocarbazolyl, diazocarbazolyl, and quinazolinyl, optionally pyridyl, pyrimidinyl, triazinyl, dibenzofuryl, dibenzothienyl, azodibenzofuryl, azodibenzothienyl, diazodibenzofuryl, diazodibenzothienyl, carbazolyl, azocarbazolyl, and diazocarbazolyl.

The following examples are merely described to facilitate the understanding of the technical invention, and should not be considered as specific limitations of the present invention.

All raw materials, solvents and the like involved in the synthesis of componnds in the present invention are purchased from Alfa, Acros, and other suppliers known to persons skilled in the art.

### Synthesis of a Compound La002

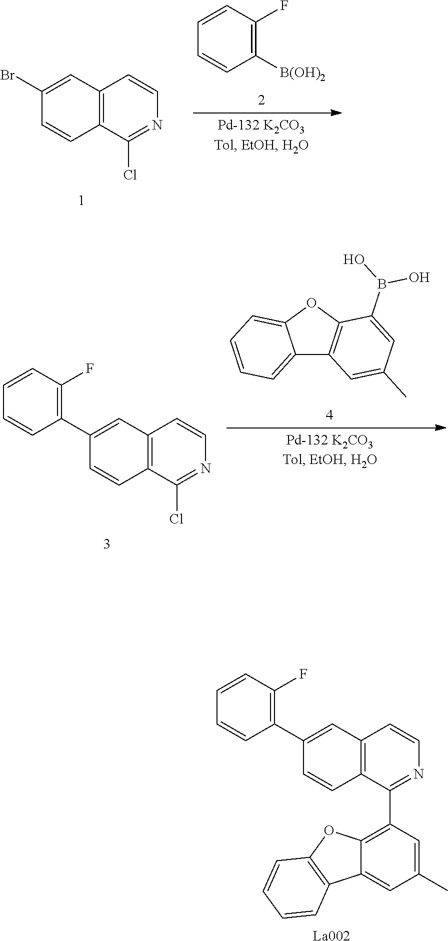

### Synthesis of an Intermediate 3

A raw material 1 (30.00 g, 123.7 mmol, 1.0 eq), a raw material 2 (20.76 g, 148.4mmol, 1.2 eq), Pd-132 (439.2 mg, 0.61 mmol, 0.005 eq), potassium carbonate (34.2 g, 247.2 mmol, 2.0 eq), toluene (300 ml), ethanol (90 ml) and deionized water (90 ml) were added into a 1 L three-neck flask, subjected to vacuumizing for nitrogen replacement for 3 times, and stirred under the protection of nitrogen at 60° C. for 1 h. According to monitoring by thin-layer chromatography (TLC), the raw material 1 was completely reacted. After cooling to room temperature, a reaction solution was subjected to liquid separation, an organic phase was collected and washed with deionized water for 2 times (100 ml/time), then the organic phase was filtered with silica gel and rinsed with toluene (50 ml), and a filtrate was collected and spin-dried to obtain a solid. Then, the solid was recrystallized with tetrahydrofuran (60 ml) and ethanol (150 ml) at 5° C. for 1 time, and a solid was collected by filtration and then dried to obtain a white solid intermediate 3 (22.3 g, yield: 69.95%). Mass spectrometry was performed at 258.69 (M+H).

### Synthesis of a Compound La002

The intermediate 3 (22.00 g, 85.37 mmol, 1.0 eq), a raw material 4 (23.16 g, 102.45 mmol, 1.2 eq), Pd-132 (604.51 mg, 0.85 mmol, 0.01 eq), potassium carbonate (23.6 g, 170.75 mmol, 2.0 eq), toluene (300 ml), ethanol (100 ml) and deionized water (100 ml) were added into a 1 L three-neck flask, subjected to vacuumizing for nitrogen replacement for 3 times, and stirred under the protection of nitrogen at 65° C. for 2 h. According to monitoring by TLC, the raw material 3 was completely reacted. After cooling to room temperature, a reaction solution was subjected to liquid separation, an organic phase was collected and washed with deionized water for 2 times (200 ml/time), then the organic phase was filtered with silica gel and rinsed with toluene (100 ml), and a filtrate was collected and spin-dried to obtain a solid. Then, the solid was recrystallized with tetrahydrofuran (200 ml) and ethanol (200 ml) at room temperature for 2 times, and a solid was collected by filtration and then dried to obtain a white solid compound La002 (24.0 g, yield: 69.68%). Mass spectrometry was performed at 404.45 (M+H). ¹HNMR (400 MHz, CDCl₃) δ8.75 (d, J=5.7 Hz, 1H), 8.11 (s, 1H), 8.00 (d, J=7.4 Hz, 1H), 7.96-7.87 (m, 2H), 7.81 (d, J=5.6 Hz, 1H), 7.69 (d, J=8.8 Hz, 1H), 7.60-7.52 (m, 2H), 7.46-7.31 (m, 4H), 7.26 (ddd, J=26.3, 13.3, 4.7 Hz, 2H), 2.62 (s, 3H).

### Synthesis of a Compound Ir(La002)₂Lb005

### Synthesis of a Compound Ir(La002)-1

The compound La002 (17.22 g, 42.68 mmol, 3.5 eq) and IrCl₃·3H₂O (4.30 g, 12.19 mmol, 1.0 eq) were put into a 500 ml one-neck round-bottomed flask, ethylene glycol ethyl ether (260 ml) and deionized water (86 ml) were added, and a resulting mixture was subjected to vacuumizing for replacement for 3 times and stirred under the protection of N₂ at 110° C. for 20 h. After cooling to room temperature, methanol (130 ml) was added and stirred for 1 h, and filtration was performed to collect a solid so as to obtain a dark red solid compound Ir (La002)-1 (10.23 g, 81.25%). The obtained compound was directly used in the next step without further purification.

### Synthesis of a Compound Ir(La002)₂Lb005

The compound Ir(La002)-1 (10.23 g, 9.91 mmol, 1.0 eq), Lb005 (10.50 g, 49.54 mmol, 5.0 eq) and sodium carbonate (10.50 g, 99.08 mmol, 10.0 eq) were put into a 500 ml one-neck round-bottomed flask, ethylene glycol ethyl ether (200 ml) was added, and a resulting mixture was subjected to vacuumizing for replacement for 3 times and stirred under the protection of N₂ at 50° C. for 24 h. According to monitoring by TLC, the Ir(La002)-1 was completely reacted. After cooling to room temperature, 250 ml of methanol was added for beating at room temperature for 2 h, suction filtration was performed, a filter cake was dissolved in dichloromethane (330 ml) and filtered with silica gel, and a filtrate was washed with deionized water (120 ml) for 3 times. Then, liquid separation was performed, an organic phase was collected, concentrated and dried to obtain a dark red solid, and the solid was recrystallized with tetrahydrofuran and methanol (7V/4V) for three times to obtain a red solid compound Ir(La002)₂Lb005(6.22 g, yield: 51.95%). 6.22 g of the crude product Ir(La002)₂Lb005 was sublimated and purified to obtain sublimated and purified Ir(La002)₂Lb005 (3.34 g, yield: 53.69%). Mass spectrometry was performed at 1209.42 (M+H). ¹HNMR (400 MHZ, CDCl₃) δ9.08 (d, J=9.0 Hz, 2H), 8.35 (d, J=6.3 Hz, 2H), 8.04 (s, 2H), 7.91 (d, J=8.9 Hz, 2H), 7.83 (d, J=6.9 Hz, 2H), 7.70-7.65 (m, 2H), 7.50 (d, J=8.0 Hz, 2H), 7.47-7.39 (m, 6H), 7.38-7.32 (m, 4H), 7.32-7.26 (m, 4H), 4.85 (s, 1H), 1.68 (s, 6H), 1.29 (dd, J=15.2, 6.6 Hz, 3H), 1.12 (dd, J=13.0, 7.4 Hz, 2H), 0.91-0.72 (m, 5H), 0.51 (t, J=7.4 Hz, 6H), −0.11 (t, J=7.4 Hz, 6H).

### Synthesis of a Compound La005

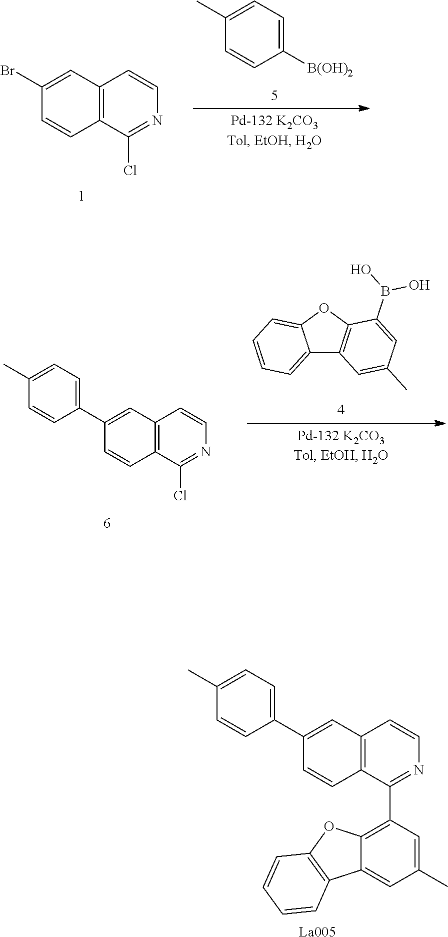

### Synthesis of an Intermediate 6

With reference to the synthesis and purification methods of the intermediate 3, only the corresponding raw materials were required to be changed, and a target compound intermediate 6 was obtained. Mass spectrometry was performed at 254.73 (M+H).

### Synthesis of a Compound La005

With reference to the synthesis and purification methods of the compound La002, only the corresponding raw materials were required to be changed, and a target compound La005 was obtained. Mass spectrometry was performed at 400.48 (M+H). ¹H NMR (400 MHZ, CDCl₃) δ8.73 (d, J=5.7 Hz, 1H), 8.10 (s, 1H), 8.01 (d, J=7.6 Hz, 1H), 7.96-7.87 (m, 2H), 7.81 (d, J=5.8 Hz, 1H), 7.74 (d, J=8.7 Hz, 1H), 7.65 (d, J=8.1 Hz, 2H), 7.56 (s, 1H), 7.47-7.30 (m, 5H), 2.63 (s, 3H), 2.44 (s, 3H).

### Synthesis of a Compound Ir(La005)₂Lb005

### Synthesis of a Compound Ir(La005)-1

With reference to the synthesis and purification methods of the compound Ir(La002)-1, only the corresponding raw materials were required to be changed, and a compound Ir(La005)-1 was obtained and directly used in the next step without purification.

### Synthesis of a Compound Ir(La005)₂Lb005

With reference to the synthesis and purification methods of the compound Ir(La002)₂Lb005, only the corresponding raw materials were required to be changed, and a red solid compound Ir(La005)₂Lb005 was obtained (4.14 g, yield: 47.93%). 4.14 g of the crude product Ir(La005)₂Lb005 was sublimated and purified to obtain sublimated and purified Ir(La005)₂Lb005 (2.31 g, yield: 55.79%). Mass spectrometry was performed at 1201.49 (M+H). ¹H NMR (400 MHZ, CDCl₃) δ9.06 (d, J=9.0 Hz, 2H), 8.32 (d, J=6.3 Hz, 2H), 8.02 (s, 2H), 7.95 (d, J=10.4 Hz, 2H), 7.82 (d, J=7.2 Hz, 2H), 7.75 (d, J=8.0 Hz, 4H), 7.49 (d, J=8.2 Hz, 2H), 7.42-7.26 (m, 12H), 4.84 (s, 1H), 2.47 (s, 6H), 1.68 (s, 6H), 1.38-1.20 (m, 4H), 1.11 (dd, J=13.0, 7.4 Hz, 2H), 0.81 (dd, J=14.5, 8.0 Hz, 4H), 0.50 (t, J=7.4 Hz, 6H), −0.14 (t, J=7.4 Hz, 6H).

### Synthesis of a Compound La018

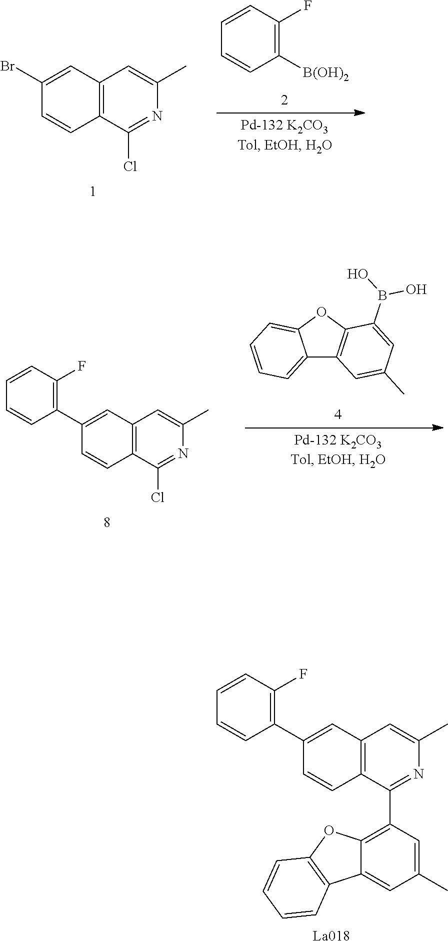

### Synthesis of an Intermediate 8

With reference to the synthesis and purification methods of the intermediate 3, only the corresponding raw materials were required to be changed, and a target compound intermediate 8 was obtained. Mass spectrometry was performed at 272.72 (M+H).

### Synthesis of a Compound La018

With reference to the synthesis and purification methods of the compound La002, only the corresponding raw materials were required to be changed, and a target compound La018 was obtained. Mass spectrometry was performed at 418.47 (M+H).

### Synthesis of a Compound Ir(La018)₂Lb005

### Synthesis of a Compound Ir(La018)-1

With reference to the synthesis and purification methods of the compound Ir(La002)-1, only the corresponding raw materials were required to be changed, and a compound Ir(La018)-1 was obtained and directly used in the next step without purification.

### Synthesis of a Compound Ir(La018)₂Lb005

With reference to the synthesis and purification methods of the compound Ir(La002)₂Lb005, only the corresponding raw materials were required to be changed, and a red solid compound Ir(La018)₂Lb005 was obtained (5.04 g, yield: 53.74%). 5.04 g of the crude product Ir(La018)₂Lb005 was sublimated and purified to obtain sublimated and purified Ir(La018)₂Lb005 (2.63 g, yield: 52.18%). Mass spectrometry was performed at 1237.47 (M+H). ¹H NMR (400 MHZ, CDCl₃) δ8.96 (d, 2H), 8.37 (d, 2H), 7.85 (s, 2H), 7.54 (m,6H), 7.44 (m, 2H), 7.42-7.23 (m, 12H), 4.83 (s, 1H), 3.71 (s, 2H), 2.69 (s, 6H), 2.34 (s, 6H), 1.27 (d, J=35.0 Hz, 8H), 1.07-0.89 (m, 12H).

### Synthesis of a Compound La025

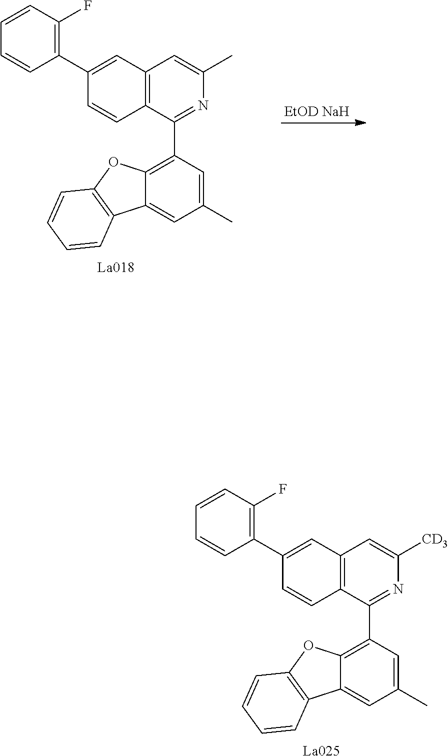 [SMILES: "EtOD NaH" ; Cc1cc(-c2nc(C)cc3cc(-c4ccccc4F)ccc23)c2oc3ccccc3c2c1 ; "La018" ; [2H]C([2H])([2H])c1cc2cc(-c3ccccc3F)ccc2c(-c2cc(C)cc3c2oc2ccccc23)n1 ; "La025"]

The compound La018 (9.32 g, 22.32 mmol, 1.0 eq), 60% sodium hydride (2.68 g, 66.97 mmol, 3.0 eq) and deuterated ethanol (93 ml) were put into a 1 L one-neck flask. A resulting mixture was subjected to vacuumizing for nitrogen replacement for three times and heated to 75° C. to carry out a reaction under the protection of nitrogen for 16 h. The reaction temperature was lowered to room temperature. Heavy water (40 mL) was added and stirred to precipitate out a solid, and the solid was collected by filtration. The crude product was separated by silica gel column chromatography (with a mixture of dichloromethane and n-hexane at a ratio of 1:15 as an eluting agent) to obtain a white solid compound La025 (6.82 g, yield: 72.64%). Mass spectrometry was performed at 421.49 (M+H).

### Synthesis of a Compound Ir(La025)₂Lb005

### Synthesis of a Compound Ir(La025)-1

With reference to the synthesis and purification methods of the compound Ir(La002)-1, only the corresponding raw materials were required to be changed, and a compound Ir(La025)-1 was obtained and directly used in the next step without purification.

### Synthesis of a Compound Ir(La025)₂Lb005

With reference to the synthesis and purification methods of the compound Ir(La002)₂Lb005, only the corresponding raw materials were required to be changed, and a red solid compound Ir(La025)₂Lb005 was obtained (5.04 g, yield: 53.74%). 5.04 g of the crude product Ir(La025)₂Lb005 was sublimated and purified to obtain sublimated and purified Ir(La025)₂Lb005 (2.63 g, yield: 52.18%). Mass spectrometry was performed at 1243.51 (M+H). ¹H NMR (400 MHZ, CDCl₃) δ8.99 (d, 2H), 8.38 (d, 2H), 7.85 (s, 2H), 7.59 (m, 6H), 7.45 (m, 2H), 7.44-7.25 (m, 12H), 4.84 (s, 1H), 3.71 (s, 2H), 2.37 (s, 6H), 1.27 (d, J=35.0 Hz, 8H), 1.07-0.89 (m, 12H).

### Synthesis of a Compound La031

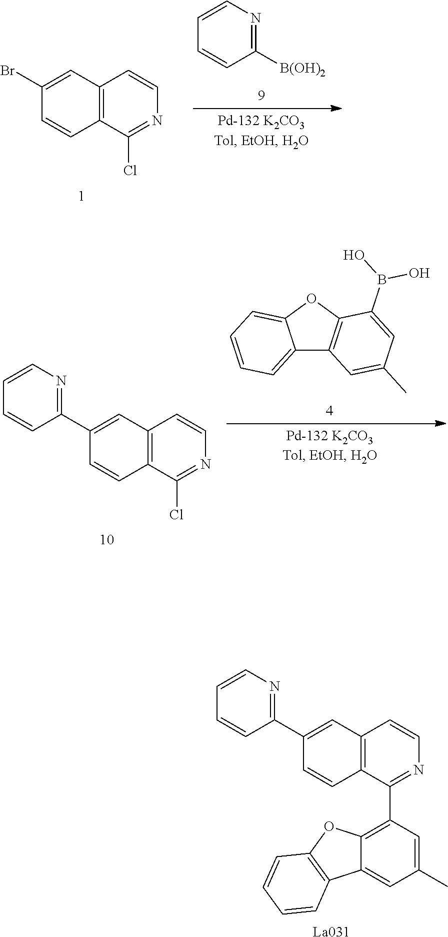 [SMILES: OB(O)c1ccccn1 ; Clc1nccc2cc(Br)ccc12 ; Clc1nccc2cc(-c3ccccn3)ccc12 ; Cc1cc(B(O)O)c2oc3ccccc3c2c1 ; Cc1cc(-c2nccc3cc(-c4ccccn4)ccc23)c2oc3ccccc3c2c1 ; "La031"]

### Synthesis of an Intermediate 10

With reference to the synthesis and purification methods of the intermediate 3, only the corresponding raw materials were required to be changed, and a target compound intermediate 10 was obtained. Mass spectrometry was performed at 241.69 (M+H).

### Synthesis of a Compound La031

With reference to the synthesis and purification methods of the compound La002, only the corresponding raw materials were required to be changed, and a target compound La031 was obtained. Mass spectrometry was performed at 387.44 (M+H).

### Synthesis of a Compound Ir(La031)₂Lb005

### Synthesis of a Compound Ir(La031)-1

With reference to the synthesis and purification methods of the compound Ir(La002)-1, only the corresponding raw materials were required to be changed, and a compound Ir(La031)-1 was obtained and directly used in the next step without

### Synthesis of a Compound Ir(La031)₂Lb005

With reference to the synthesis and purification methods of the compound Ir(La002)₂Lb005, only the corresponding raw materials were required to be changed, and a red solid compound Ir(La031)₂Lb005 was obtained (4.59 g, yield: 44.87%). 4.59 g of the crude product Ir(La031)₂Lb005 was sublimated and purified to obtain sublimated and purified Ir(La031)₂Lb005 (2.12 g, yield: 46.18%). Mass spectrometry was performed at 1175.4 (M+H). ¹H NMR (400 MHZ, CDCl₃) δ8.93 (d, 2H), 8.37 (d, 2H), 8.23 (d, 2H), 8.11 (d, 2H), 7.98 (m, 2H), 7.56 (d, J=15.0 Hz, 4H), 7.45-7.26 (m, 6H), 7.14 (m, 4H), 6.90 (m, 4H), 4.81 (s, 1H),2.34 (s, 6H), 1.27 (d, J=35.0 Hz, 6H), 1.07-0.84 (m, 16H).

### Synthesis of a Compound La032

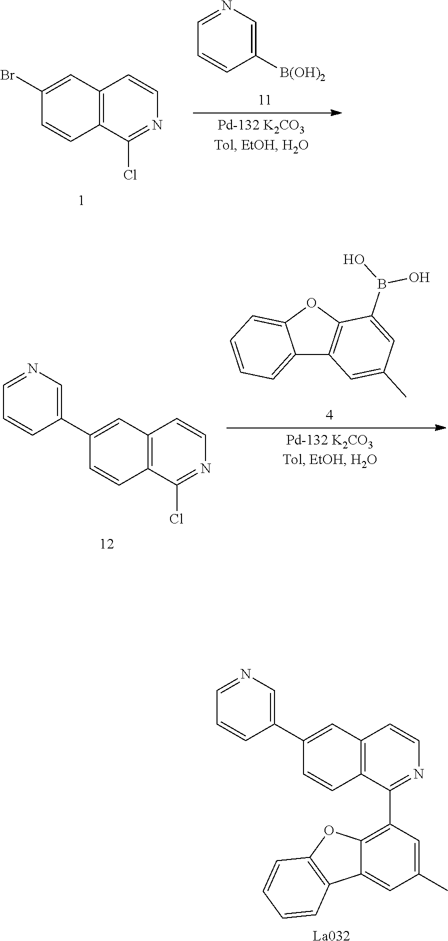 [SMILES: OB(O)c1cccnc1 ; Clc1nccc2cc(Br)ccc12 ; Clc1nccc2cc(-c3cccnc3)ccc12 ; Cc1cc(B(O)O)c2oc3ccccc3c2c1 ; Cc1cc(-c2nccc3cc(-c4cccnc4)ccc23)c2oc3ccccc3c2c1 ; "La032"]

### Synthesis of an Intermediate 12

With reference to the synthesis and purification methods of the intermediate 3, only the corresponding raw materials were required to be changed, and a target compound intermediate 12 was obtained. Mass spectrometry was performed at 241.69 (M+H).

### Synthesis of a Compound La032

With reference to the synthesis and purification methods of the compound La002, only the corresponding raw materials were required to be changed, and a target compound La032 was obtained. Mass spectrometry was performed at 387.44 (M+H).

### Synthesis of a Compound Ir(La032)₂Lb005

### Synthesis of a Compound Ir(La032)-1

With reference to the synthesis and purification methods of the compound Ir(La002)-1, only the corresponding raw materials were required to be changed, and a compound Ir(La032)-1 was obtained and directly used in the next step without purification.

### Synthesis of a ComponndIr(La032)₂Lb005

With reference to the synthesis and purification methods of the compound Ir(La002)₂Lb005, only the corresponding raw materials were required to be changed, and a red solid compound Ir(La032)₂Lb005 was obtained (4.17 g, yield: 46.31%). 4.17 g of the crude product Ir(La032)₂Lb005 was sublimated and purified to obtain sublimated and purified Ir(La032)₂Lb005 (1.94 g, yield: 46.52%). Mass spectrometry was performed at 1175.4 (M+H). ¹H NMR (400 MHZ, CDCl₃) 9.24 (d, 2H), 8.70 (d, 2H), 8.48 (d, 2H), 8.33 (d, 2H), 8.11 (m, 2H), 7.98 (m, 2H), 7.84 (m, 6H), 7.61-7.44 (m, 6H), 7.35 (d, J=40.0 Hz, 4H), 4.82 (s, 1H),2.34 (s, 6H), 1.28 (d, J=35.0 Hz, 6H), 1.08-0.85 (m, 16H).

### Synthesis of a Compound La033

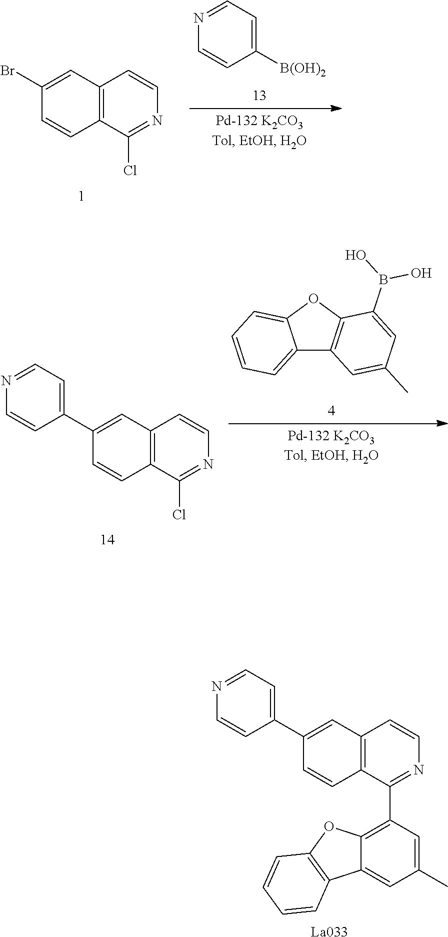 [SMILES: OB(O)c1ccncc1 ; Clc1nccc2cc(Br)ccc12 ; Clc1nccc2cc(-c3ccncc3)ccc12 ; Cc1cc(B(O)O)c2oc3ccccc3c2c1 ; Cc1cc(-c2nccc3cc(-c4ccncc4)ccc23)c2oc3ccccc3c2c1 ; "La033"]

### Synthesis of an Intermediate 14

With reference to the synthesis and purification methods of the intermediate 3, only the corresponding raw materials were required to be changed, and a target compound intermediate 14 was obtained. Mass spectrometry was performed at 241.69 (M+H).

### Synthesis of a Compound La033

With reference to the synthesis and purification methods of the compound La002, only the corresponding raw materials were required to be changed, and a target compound La033 was obtained. Mass spectrometry was performed at 387.44 (M+H).

### Synthesis of a Compound Ir(La033)₂Lb005

### Synthesis of a Compound Ir(La033)-1

With reference to the synthesis and purification methods of the compound Ir(La002)-1, only the corresponding raw materials were required to be changed, and a compound Ir(La033)-1 was obtained and directly used in the next step without purification.

### Synthesis of a Compound Ir(La033)₂Lb005

With reference to the synthesis and purification methods of the compound Ir(La002)₂Lb005, only the corresponding raw materials were required to be changed, and a red solid compound Ir(La033)₂Lb005 was obtained (4.17 g, yield: 46.31%). 4.17 g of the crude product Ir(La033)₂Lb005 was sublimated and purified to obtain sublimated and purified Ir(La033)₂Lb005 (1.94 g, yield: 46.52%). Mass spectrometry was performed at 1175.4 (M+H). ¹H NMR (400 MHZ, CDCl₃) δ9.01 (d, 2H), 8.52 (d, 2H), 8.24 (d, 2H), 8.12 (d, 2H), 7.96 (m, 2H), 7.57 (d, J=15.0 Hz, 4H), 7.45-7.26 (m, 6H), 7.17 (m, 4H), 6.92 (m, 4H), 4.82 (s, 1H),2.34 (s, 6H), 1.28 (d, J=35.0 Hz, 6H), 1.08-0.85 (m, 16H).

### Synthesis of a Compound La042

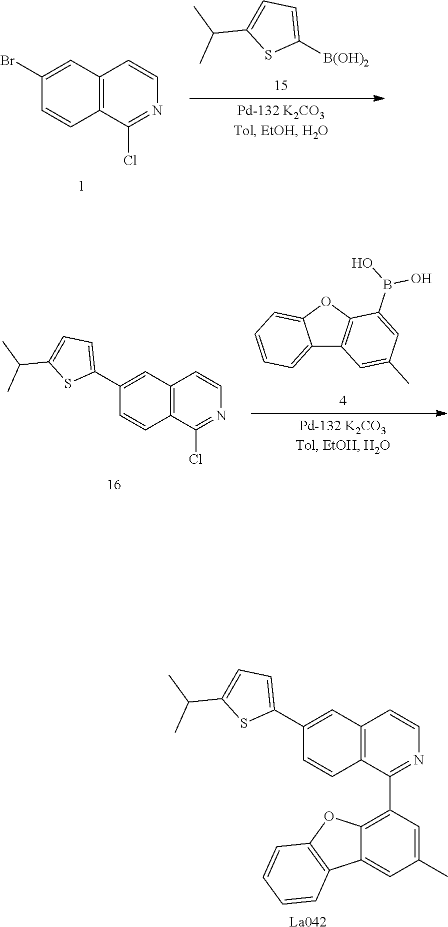

### Synthesis of an Intermediate 16

With reference to the synthesis and purification methods of the intermediate 3, only the corresponding raw materials were required to be changed, and a target compound intermediate 16 was obtained. Mass spectrometry was performed at 288.81 (M+H).

### Synthesis of a Compound La042

With reference to the synthesis and purification methods of the compound La002, only the corresponding raw materials were required to be changed, and a target compound La042 was obtained. Mass spectrometry was performed at 434.56 (M+H).

### Synthesis of a Componnd Ir(La042)₂Lb005

### Synthesis of a Compound Ir(La042)-1

With reference to the synthesis and purification methods of the compound Ir(La002)-1, only the corresponding raw materials were required to be changed, and a compound Ir(La042)-1 was obtained and directly used in the next step without purification.

### Synthesis of a Componnd Ir(La042)₂Lb005

With reference to the synthesis and purification methods of the compound Ir(La002)₂Lb005, only the corresponding raw materials were required to be changed, and a red solid compound Ir(La042)₂Lb005 was obtained (4.39 g, yield: 50.32%). 4.39 g of the crude product Ir(La042)₂Lb005 was sublimated and purified to obtain sublimated and purified Ir(La042)₂Lb005 (2.35 g, yield: 53.53%). Mass spectrometry was performed at 1269.65 (M+H). ¹H NMR (400 MHZ, CDCl₃) 8.97 (d, 2H), 8.38 (d, 2H), 7.98 (d, 2H), 7.84 (d, 2H), 7.56 (d, J=15.0 Hz, 4H), 7.39 (m, 4H), 7.31 (m, 4H), 6.71 (d,4H), 4.79 (s, 1H), 3.10 (m, 2H), 2.34 (s, 6H), 1.31 (m, 4H), 1.22 (m, 14H), 1.07-0.88 (m, 16H).

### Synthesis of a Compound La050

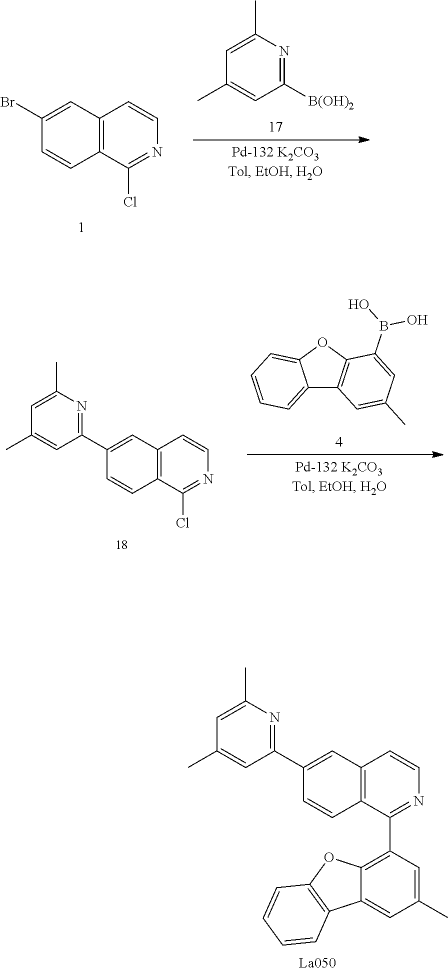

### Synthesis of an Intermediate 18

With reference to the synthesis and purification methods of the intermediate 3, only the corresponding raw materials were required to be changed, and a target compound intermediate 18 was obtained. Mass spectrometry was performed at 269.74 (M+H).

### Synthesis of a Compound La050

With reference to the synthesis and purification methods of the compound La002, only the corresponding raw materials were required to be changed, and a target compound La050 was obtained. Mass spectrometry was performed at 415.50 (M+H).

### Synthesis of a Compound Ir(La050)₂Lb005

### Synthesis of a Compound Ir(La050)-1

With reference to the synthesis and purification methods of the compound Ir(La002)-1, only the corresponding raw materials were required to be changed, and a compound Ir(La050)-1 was obtained and directly used in the next step without purification.

### Synthesis of a Compound Ir(La050)₂Lb005

With reference to the synthesis and purification methods of the compound Ir(La002)₂Lb005, only the corresponding raw materials were required to be changed, and a red solid compound Ir(La050)₂Lb005 was obtained (3.82 g, yield: 43.67%). 3.82 g of the crude product Ir(La050)₂Lb005 was sublimated and purified to obtain sublimated and purified Ir(La050)₂Lb005 (1.74 g, yield: 45.54%). Mass spectrometry was performed at 1231.52 (M+H). ¹H NMR (400 MHZ, CDCl₃) δ8.96 (d, 2H), 8.23 (d, 2H), 8.11 (d, 2H), 7.98 (d, 2H), 7.68 (s, 2H), 7.56 (m, 4H), 7.39 (m, 4H), 7.31 (m, 4H), 6.99 (s, 2H), 4.83 (s, 1H), 2.68 (s, 6H), 2.38 (d, J=40.0 Hz, 12H), 1.27 (m, 6H), 1.07-0.85 (m, 16H).

### Synthesis of a Compound La068

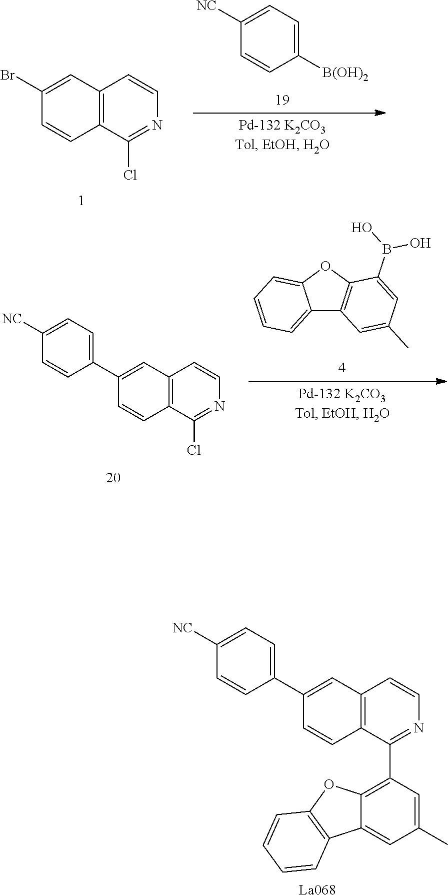 [SMILES: N#Cc1ccc(B(O)O)cc1 ; Clc1nccc2cc(Br)ccc12 ; N#Cc1ccc(-c2ccc3c(Cl)nccc3c2)cc1 ; Cc1cc(B(O)O)c2oc3ccccc3c2c1 ; Cc1cc(-c2nccc3cc(-c4ccc(C#N)cc4)ccc23)c2oc3ccccc3c2c1 ; "La068"]

### Synthesis of an Intermediate 20

With reference to the synthesis and purification methods of the intermediate 3, only the corresponding raw materials were required to be changed, and a target compound intermediate 20 was obtained. Mass spectrometry was performed at 265.71 (M+H).

### Synthesis of a Compound La068

With reference to the synthesis and purification methods of the compound La002, only the corresponding raw materials were required to be changed, and a target compound La068 was obtained. Mass spectrometry was performed at 411.47 (M+H).

### Synthesis of a Compound Ir(La068)₂Lb005

### Synthesis of a Compound Ir(La068)-1

With reference to the synthesis and purification methods of the compound Ir(La002)-1, only the corresponding raw materials were required to be changed, and a compound Ir(La068)-1 was obtained and directly used in the next step without purification.

### Synthesis of a Compound Ir(La068)₂Lb005

With reference to the synthesis and purification methods of the compound Ir(La002)₂Lb005, only the corresponding raw materials were required to be changed, and a red solid compound Ir(La068)₂Lb005 was obtained (3.24 g, yield: 41.61%). 3.24 g of the crude product Ir(La068)₂Lb005 was sublimated and purified to obtain sublimated and purified Ir(La068)₂Lb005 (1.86 g, yield: 57.40%). Mass spectrometry was performed at 1223.45 (M+H). ¹H NMR (400 MHZ, CDCl₃) 89.02 (d, 2H), 8.43 (d, 2H), 7.95 (m, 6H), 7.84 (m, 4H), 7.53 (t, J=12.5 Hz, 6H), 7.35 (m, 8H), 4.83 (s, 1H), 2.34 (s, 6H), 1.27 (m, 6H), 1.08-0.85 (m, 16H).

### Synthesis of a Compound La079

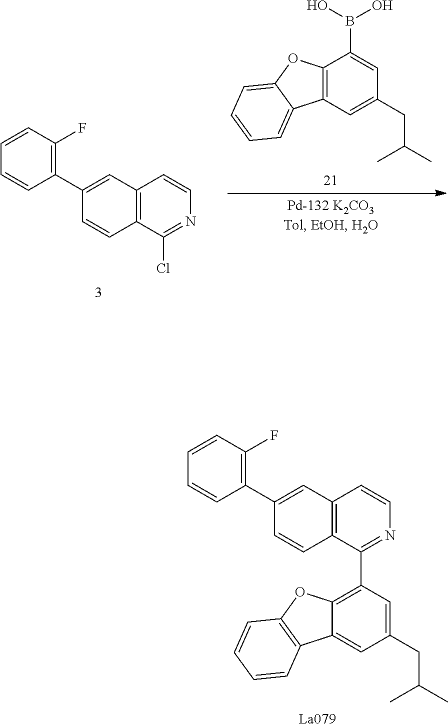 [SMILES: Fc1ccccc1-c1ccc2c(Cl)nccc2c1 ; CC(C)Cc1cc(B(O)O)c2oc3ccccc3c2c1 ; CC(C)Cc1cc(-c2nccc3cc(-c4ccccc4F)ccc23)c2oc3ccccc3c2c1 ; "La079"]

With reference to the synthesis and purification methods of the compound La002, only the corresponding raw materials were required to be changed, and a target compound La079 was obtained. Mass spectrometry was performed at 446.53 (M+H).

### Synthesis of a Compound Ir(La079)₂Lb005

### Synthesis of a Compound Ir(La079)-1

With reference to the synthesis and purification methods of the compound Ir(La002)-1, only the corresponding raw materials were required to be changed, and a compound Ir(La079)-1 was obtained and directly used in the next step without purification.

### Synthesis of a Compound Ir(La079)₂Lb005

With reference to the synthesis and purification methods of the compound Ir(La002)₂Lb005, only the corresponding raw materials were required to be changed, and a red solid compound Ir(La079)₂Lb005 was obtained (2.77 g, yield: 41.61%). 2.77 g of the crude product Ir(La079)₂Lb005 was sublimated and purified to obtain sublimated and purified Ir(La079)₂Lb005 (1.75 g, yield: 63.17%). Mass spectrometry was performed at 1293.58 (M+H). ¹HNMR (400 MHZ, CDCl₃) δ9.08 (d, J=9.0 Hz, 2H), 8.35 (d, J=6.3 Hz, 2H), 8.04 (s, 2H), 7.91 (d, J=8.9 Hz, 2H), 7.83 (d, J=6.9 Hz, 2H), 7.70-7.65 (m, 2H), 7.50 (d, J=8.0 Hz, 2H), 7.47-7.39 (m, 6H), 7.38-7.32 (m, 4H), 7.32-7.26 (m, 4H), 4.85 (s, 1H), 2.67 (m,2H),2.21 (d,4H), 1.36 (s, 12H), 1.29 (dd, J=15.2, 6.6 Hz, 3H), 1.12 (dd, J=13.0, 7.4 Hz, 2H), 0.91-0.72 (m, 5H), 0.51 (t, J=7.4 Hz, 6H), −0.11 (t, J=7.4 Hz, 6H).

### Synthesis of a Compound La086

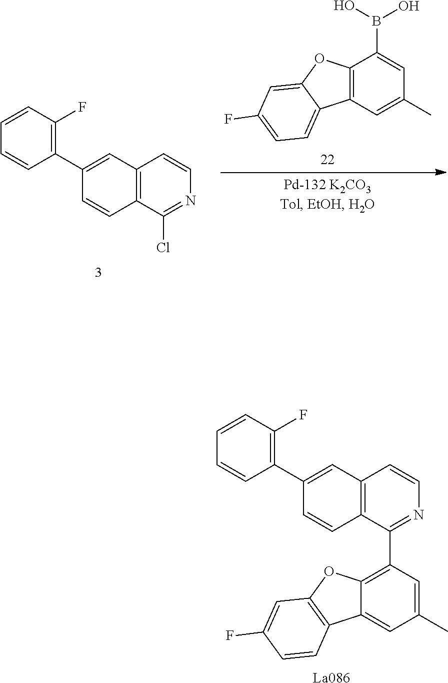 [SMILES: Fc1ccccc1-c1ccc2c(Cl)nccc2c1 ; Cc1cc(B(O)O)c2oc3cc(F)ccc3c2c1 ; Cc1cc(-c2nccc3cc(-c4ccccc4F)ccc23)c2oc3cc(F)ccc3c2c1 ; "La086"]

With reference to the synthesis and purification methods of the compound La002, only the corresponding raw materials were required to be changed, and a target compound La079 was obtained. Mass spectrometry was performed at 422.44 (M+H).

### Synthesis of a Compound Ir(La086)₂Lb005

Synthesis of a compound Ir (La086)-1

With reference to the synthesis and purification methods of the compound Ir(La002)-1, only the corresponding raw materials were required to be changed, and a compound Ir(La086)-1 was obtained and directly used in the next step without purification.

### Synthesis of a Compound Ir(La086)₂Lb005

With reference to the synthesis and purification methods of the compound Ir(La002)₂Lb005, only the corresponding raw materials were required to be changed, and a red solid compound Ir(La086)₂Lb005 was obtained (2.64 g, yield: 40.77%). 2.63 g of the crude product Ir(La086)₂Lb005 was sublimated and purified to obtain sublimated and purified Ir(La086)₂Lb005 (1.56 g, yield: 59.31%). Mass spectrometry was performed at 1245.44 (M+H). ¹HNMR (400 MHZ, CDCl₃) δ9.02 (d, J=9.1 Hz, 2H), 8.31 (d, J=6.6 Hz, 2H), 8.02 (s, 2H), 7.88 (d, J=8.7 Hz, 2H), 7.81 (d, J=6.6 Hz, 2H), 7.72-7.62 (m, 2H), 7.49-7.36 (m, 6H), 7.35-7.32 (m, 4H), 7.31-7.26 (m, 4H), 4.85 (s, 1H), 1.68 (s, 6H), 1.28 (dd, J=15.2, 6.6 Hz, 3H), 1.13 (dd, J=13.0, 7.4 Hz, 2H), 0.93-0.71 (m, 5H), 0.52 (t, J=7.4 Hz, 6H), −0.12 (t, J=7.4 Hz, 6H).

### Synthesis of a Compound Ir(La005)₂Lb009

With reference to the synthesis and purification methods of the compound Ir(La002)₂Lb005, only the corresponding raw materials were required to be changed, and a red solid compound Ir(La005)₂Lb009 was obtained (4.12 g, yield: 50.37%). 4.12 g of the crude product Ir(La005)₂Lb009 was sublimated and purified to obtain sublimated and purified Ir(La005)₂Lb009 (2.52 g, yield: 61.16%). Mass spectrometry was performed at 1197.46 (M+H). ¹H NMR (400 MHZ, CDCl₃) 9.03 (d, J=9.0 Hz, 2H), 8.35 (d, J=6.3 Hz, 2H), 8.01 (s, 2H), 7.96 (d, J=10.4 Hz, 2H), 7.85 (d, J=7.2 Hz, 2H), 7.73 (d, J=8.0 Hz, 4H), 7.51 (d, J=8.2 Hz, 2H), 7.43-7.27 (m, 12H), 4.84 (s, 1H),2.35 (m, 13H), 2.20 (m, 2H), 1.65 (m, 12H), 1.34 (m, 6H).

### Synthesis of a Compound Ir(La005)₂Lb018

With reference to the synthesis and purification methods of the compound Ir(La002)₂Lb005, only the corresponding raw materials were required to be changed, and a red solid compound Ir(La005)₂Lb018 was obtained (3.68 g, yield: 53.14%). 3.68 g of the crude product Ir(La005)₂Lb018 was sublimated and purified to obtain sublimated and purified Ir(La005)₂Lb018 (2.43 g, yield: 66.03%). Mass spectrometry was performed at 1281.62 (M+H). ¹H NMR (400 MHZ, CDCl₃) 9.03 (d, J=9.0 Hz, 2H), 8.35 (d, J=6.3 Hz, 2H), 8.01 (s, 2H), 7.96 (d, J=10.4 Hz, 2H), 7.85 (d, J=7.2 Hz, 2H), 7.73 (d, J=8.0 Hz, 4H), 7.51 (d, J=8.2 Hz, 2H), 7.43-7.27 (m, 12H), 4.84 (s, 1H), 3.05 (m, 8H), 2.45 (s, 6H), 2.34 (s, 6H), 1.47 (m, 2H), 1.01 (d, J=15.0 Hz, 11H), 0.87 (s, 12H).

### Synthesis of a Compound Lc003

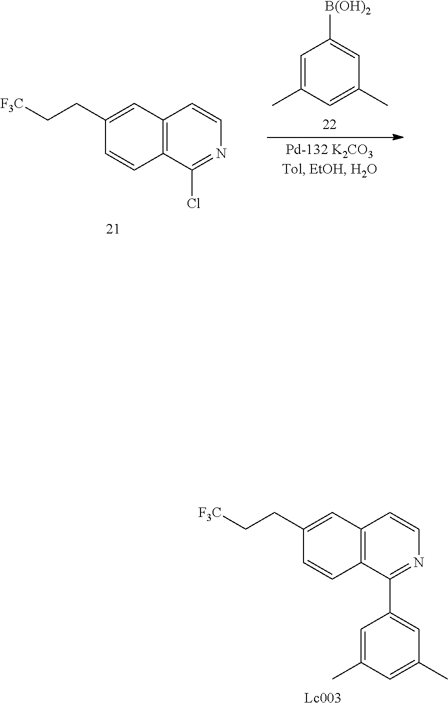

With reference to the synthesis and purification methods of the compound La002, only the corresponding raw materials were required to be changed, and a target compound Lc003 was obtained. Mass spectrometry was performed at 330.36 (M+H).

### Synthesis of a Compoud Ir(La005)(Lb009)(Lc003)

### Synthesis of a Compound Ir(La005)-2

The dimer Ir(La005)-1 (9.85 g, 9.75 mmol, 1.0 eq) and dichloromethane (740 ml) were added into a 3 L three-neck flask and stirred for dissolution. Silver trifluoromethanesulfonate (5.01 g, 19.49 mmol, 2.0 eq) was dissolved in methanol (500 ml) and then added into the original reaction solution flask, and a resulting mixture was subjected to vacuumizing for replacement for 3 times and stirred at room temperature for 16 h under the protection of N₂. Then, a reaction solution was filtered with diatomite, a filter residue was rinsed with dichloromethane (200 ml), and a filtrate was spin-dried to obtain a compound Ir(La005)-2 (7.82 g, yield: 76.21%). The obtained compound was directly used in the next step without purification.

### Synthesis of a Compound Ir(La005)₂Lc003

The compound Ir(La005)-2 (7.8 g, 7.41 mmol, 1.0 eq) and the Lc003 (6.1 g, 18.53 mmol, 2.5 eq) were added into a 250 ml three-neck flask, ethanol (80 ml) was added, vacuumizing was performed for replacement for 3 times, and stirring was performed for reflux for 16 h under the protection of N₂. After cooling to room temperature, filtration was performed, a solid was collected, dissolved in dichloromethane (220 ml) and filtered with silica gel, and a filter cake was rinsed with dichloromethane (80 ml). Then, a filtrate was spin-dried, recrystallized with tetrahydrofuran/methanol (the ratio of the product to tetrahydrofuran to methanol was 1:7:10) for 2 times, and dried to obtain a compound Ir(La005)₂Lc003 (4.51 g, 46.2%). Mass spectrometry was performed at 1318.52 (M+H).

### Synthesis of a Compound Ir(La005)₂ (Lc003)-1

The compound Ir(La005)₂Lc003 (6.33 g, 4.80 mmol, 1.0 eq) and zinc chloride (32.74 g, 240.22 mmol, 50 eq) were put into a 1 L one-neck flask, 1,2-dichloroethane (380 ml) was added, and a resulting mixture was subjected to vacuumizing for replacement for 3 times and stirred to carry out a reflux reaction under the protection of N₂ for 18 h. According to monitoring by a TLC point plate, the raw material Ir(La005)₂Lc003 was basically and completely reacted. After cooling to room temperature, deionized water was added for washing for 3 times (120 ml/time). Then, a filtrate was spin-dried to obtain a compound Ir(La005)₂Lc003-1 (3.62 g, 78.84%). The obtained compound was directly used in the next step without purification.

### Synthesis of a Compound Ir(La005)(Lb009)(Lc003)

The compound Ir(La005)₂ (Lc003)-1 (3.52 g, 3.69 mmol, 1.0 eq), Lb009 (3.84 g, 18.44 mmol, 5.0 eq), and sodium carbonate (3.91 g, 36.88 mmol, 10.0 eq) were put into a 250 ml one-neck round-bottomed flask, ethylene glycol ethyl ether (56 ml) was added, and a resulting mixture was subjected to vacuumizing for replacement for 3 times and stirred under the protection of N₂ at 50° C. for 24 h. According to monitoring by TLC, the Ir(La005)₂ (Lc003)-1 was completely reacted. After cooling to room temperature, 112 ml of methanol was added for beating at room temperature for 2 h, suction filtration was performed, a filter cake was dissolved in dichloromethane (100 ml), filtered with silica gel and then rinsed with dichloromethane (50 ml), and a filtrate was collected and washed with deionized water for 3 times (60 ml/time). Then, liquid separation was performed, an organic phase was collected, concentrated and dried to obtain a dark red solid, and the solid was recrystallized with tetrahydrofuran/methanol (the ratio of the product to tetrahydrofuran to methanol was 1:8:12) for 3 times to obtain a red solid compound Ir(La005)(Lb009)(Lc003) (1.72 g, yield: 41.33%). 1.72 g of the crude product Ir(La005)(Lb009)(Lc003) was sublimated and purified to obtain sublimated and purified Ir(La005)(Lb009)(Lc003) (0.93 g, yield: 54.06%). Mass spectrometry was performed at 1127.33 (M+H). ¹H NMR (400 MHZ, CDCl₃) δ8.95 (d, 1H), 8.40 (d, 1H), 8.17 (d, 1H), 8.07 (m, 2H), 7.98 (m, 2H), 7.78 (d, 1H), 7.60-7.45 (m, 6H), 7.35 (m, 2H), 7.16 (m, 3H), 6.92 (d, 1H), 4.82 (s, 1H), 2.63 (t, 2H), 2.42-2.25 (m, 13H), 2.20 (m, 2H), 1.89 (t, 2H), 1.65 (m, 12H), 1.34 (m, 4H).

### Synthesis of a Compound Ir(La005)(Lb009)(Lc004)

### Synthesis of a Compound Ir(La005)₂Lc004

With reference to the synthesis and purification methods of the compound Ir(La005)₂Lc003, only the corresponding raw materials were required to be changed, and a target compound Ir(La005)₂Lc004 was obtained. Mass spectrometry was performed at 1278.57 (M+H).

### Synthesis of a Compound Ir(La005)₂(Lc004)-1

With reference to the synthesis and purification methods of the compound Ir(La005)₂(Lc003)-1, only the corresponding raw materials were required to be changed, and a compound Ir(La005)₂(Lc004)-1 was obtained and directly used in the next step without purification.

### Synthesis of a Compound Ir(La005)(Lb009)(Lc004)

With reference to the synthesis and purification methods of the compound Ir(La005)(Lb009)(Lc003), only the corresponding raw materials were required to be changed, and a red solid compound Ir(La005)(Lb009)(Lc004) was obtained (2.03 g, yield: 38.66%). 2.03 g of the crude product Ir(La005)(Lb009)(Lc004) was sublimated and purified to obtain sublimated and purified Ir(La005)(Lb009)(Lc004) (1.18 g, yield: 58.70%). Mass spectrometry was performed at 1087.39 (M+H). ¹H NMR (400 MHZ, CDCl₃) δ8.95 (d, 1H), 8.40 (d, 1H), 8.17 (d, 1H), 8.07 (m, 2H), 7.98 (m, 2H), 7.78 (d, 1H), 7.60-7.45 (m, 6H), 7.35 (m, 2H), 7.16(m, 3H), 6.92 (d, 1H), 4.82 (s, 1H), 2.49-2.26 (m, 15H), 2.20 (m, 2H), 1.93-1.50 (m, 13H), 1.34 (d, J=40.0 Hz, 4H), 0.87 (s, 6H).

### Synthesis of a Compound Ir(La005)(Lb009)(Lc025)

### Synthesis of a Compound Ir(La005)₂Lc025

With reference to the synthesis and purification methods of the compound Ir(La005)₂Lc003, only the corresponding raw materials were required to be changed, and a target compound Ir(La005)₂Lc025 was obtained. Mass spectrometry was performed at 1354.63 (M+H).

### Synthesis of a Compound Ir(La005)₂(Lc025)-1

With reference to the synthesis and purification methods of the compound Ir(La005)₂(Lc003)-1, only the corresponding raw materials were required to be changed, and a compound Ir(La005)₂(Lc025)-1 was obtained and directly used in the next step without purification.

### Synthesis of a Compound Ir(La005)(Lb009)(Lc025)

With reference to the synthesis and purification methods of the compound Ir(La005)(Lb009)(Lc003), only the corresponding raw materials were required to be changed, and a red solid compound Ir(La005)(Lb009)(Lc025) was obtained (1.63 g, yield: 34.65%). 1.63 g of the crude product Ir(La005)(Lb009)(Lc025) and purified obtain was sublimated to sublimated and purified Ir(La005)(Lb009)(Lc025) (0.77 g, yield: 47.23%). Mass spectrometry was performed at 1163.44 (M+H). ¹H NMR (400 MHZ, CDCl₃) δ8.93 (m, 2H), 8.40 (d, 1H), 8.17 (d, 1H), 7.98 (dd, 2H), 7.78 (m, 2H), 7.60-7.45 (m, 8H), 7.35 (m, 4H), 7.16 (m, 4H), 4.84 (s, 1H), 2.43 (d, 2H), 2.35 (m, 9H), 2.20 (m, 2H), 1.91-1.47 (m, 13H), 1.34 (m, 4H), 0.87 (s, 6H).

### Synthesis of a Compound Ir(La005)(Lb009)(Lc027)

### Synthesis of a Compound Ir(La005)₂Lc027

With reference to the synthesis and purification methods of the compound Ir(La005)₂Lc003, only the corresponding raw materials were required to be changed, and a target compound IR(La005)₂Lc027 was obtained. Mass spectrometry was performed at 1366.64 (M+H).

### Synthesis of a Compound Ir(La005)₂(Lc027)-1

With reference to the synthesis and purification methods of the compound IR(La005)₂(Lc003)-1, only the corresponding raw materials were required to be changed, and a compound IR(La005)₂ (Lc027)-1 was obtained and directly used in the next step without purification.

### Synthesis of a Compound Ir(La005)(Lb009)(Lc027)

With reference to the synthesis and purification methods of the compound IR(La005)(Lb009)(Lc003), only the corresponding raw materials were required to be changed, and a red solid compound IR(La005)(Lb009)(Lc027) was obtained (1.87 g, yield: 34.65%). 1.87 g of the crude product Ir(La005)(Lb009)(Lc027) sublimated was and purified to obtain sublimated and purified Ir(La005)(Lb009)(Lc027) (0.91 g, yield: 48.66%). Mass spectrometry was performed at 1175.45 (M+H). ¹H NMR (400 MHZ, CDCl₃) δ8.93 (m, 2H), 8.40 (d, 1H), 8.17 (d, 1H), 7.98 (dd, 2H), 7.78 (m, 2H), 7.60-7.45 (m, 8H), 7.35 (m, 4H), 7.16 (m, 4H), 4.84 (s, 1H), 2.35 (m, 9H), 2.21 (m, 1H), 1.99-1.47 (m, 20H), 1.36-0.82 (m,6H).

Other compounds can be synthesized and sublimated by selecting corresponding materials based on same or similar methods.

An ultraviolet absorption spectrum and an emission spectrum of the compound IR(La002)₂Lb005/Ir(La005)₂Lb005 of the present invention in a dichloromethane solution are shown in the figures. All the componnds of the present invention have more saturated red luminescence and a relatively narrow half-peak width, which are conducive to realizing higher luminous efficiency.

### Application Example: Manufacturing of Organic Electroluminescent Devices

A glass substrate with a size of 50 mm*50 mm*1.0 mm including an ITO (70 Å/1,000 Å/110 Å) anode electrode was ultrasonically washed in ethanol for 10 min, dried at 150° C., and then treated with N₂ plasma for 30 min. The washed glass substrate was installed on a substrate support of a vacuum evaporation device. First, compounds HTM1 and P-dopant (at a ratio of 97%: 3%) for covering the electrode were co-evaporated on the surface of the side having an anode electrode line to form a thin film having a thickness of 100 Å. Then, a layer of HTMI was evaporated to form a thin film having a thickness of about 1,720 Å, a layer of HTM2 was evaporated on the HTM1 thin film to form a thin film having a thickness of 100 Å, and a main material 1, a main material 2 and a doping compound (including a reference compound X or the compound of the present invention) were co-evaporated on the HTM2 film layer at a ratio of 48.5%: 48.5%: 3% to form a film having a thickness of 400 Å, where the ratio of the main materials to the doping material was 90%: 10%. Then, an electron transport layer (ETL) and LiQ were co-evaporated on a light-emitting layer at a ratio of 50%: 50% to reach a thickness of 350 Å. Then, Yb was evaporated on an electron transport layer material to reach a thickness of 10 Å. Finally, a layer of metal Ag was evaporated to serve as an electrode having a thickness of 150 Å.

| | | | | | Electron |
|---|---|---|---|---|---|
| | HIL | HTL | EBL | Emission layer | transport layer |
| Example | Thickness/Å | Thickness/Å | Thickness/Å | Thickness/Å | Thickness/Å |
| A1 | HTM1:NDP-9 | HTM1 | HTM2 | H1:H2:Ir(La002)₂Lb005 | ETL:LiQ |
| | 100 | 1720 | 100 | 400 | 350 |
| A2 | HTM1:NDP-9 | HTM1 | HTM2 | H1:H2:Ir(La005)₂Lb005 | ETL:LiQ |
| | 100 | 1720 | 100 | 400 | 350 |
| A3 | HTM1:NDP-9 | HTM1 | HTM2 | H1:H2:Ir(La018)₂Lb005 | ETL:LiQ |
| | 100 | 1720 | 100 | 400 | 350 |
| A4 | HTM1:NDP-9 | HTM1 | HTM2 | H1:H2:Ir(La025)₂Lb005 | ETL:LiQ |
| | 100 | 1720 | 100 | 400 | 350 |
| A5 | HTM1:NDP-9 | HTM1 | HTM2 | H1:H2:Ir(La031)₂Lb005 | ETL:LiQ |
| | 100 | 1720 | 100 | 400 | 350 |
| A6 | HTM1:NDP-9 | HTM1 | HTM2 | H1:H2:Ir(La032)₂Lb005 | ETL:LiQ |
| | 100 | 1720 | 100 | 400 | 350 |
| A7 | HTM1:NDP-9 | HTM1 | HTM2 | H1:H2:Ir(La033)₂Lb005 | ETL:LiQ |
| | 100 | 1720 | 100 | 400 | 350 |
| A8 | HTM1:NDP-9 | HTM1 | HTM2 | H1:H2:Ir(La042)₂Lb005 | ETL:LiQ |
| | 100 | 1720 | 100 | 400 | 350 |
| A9 | HTM1:NDP-9 | HTM1 | HTM2 | H1:H2:Ir(La050)₂Lb005 | ETL:LiQ |
| | 100 | 1720 | 100 | 400 | 350 |
| A10 | HTM1:NDP-9 | HTM1 | HTM2 | H1:H2:Ir(La068)₂Lb005 | ETL:LiQ |
| | 100 | 1720 | 100 | 400 | 350 |
| A11 | HTM1:NDP-9 | HTM1 | HTM2 | H1:H2:Ir(La079)₂Lb005 | ETL:LiQ |
| | 100 | 1720 | 100 | 400 | 350 |
| A12 | HTM1:NDP-9 | HTM1 | HTM2 | H1:H2:Ir(La086)₂Lb005 | ETL:LiQ |
| | 100 | 1720 | 100 | 400 | 350 |
| A13 | HTM1:NDP-9 | HTM1 | HTM2 | H1:H2:Ir(La005)₂Lb009 | ETL:LiQ |
| | 100 | 1720 | 100 | 400 | 350 |
| A14 | HTM1:NDP-9 | HTM1 | HTM2 | H1:H2:Ir(La005)₂Lb018 | ETL:LiQ |
| | 100 | 1720 | 100 | 400 | 350 |
| A15 | HTM1:NDP-9 | HTM1 | HTM2 | H1:H2:IrLa005Lb009Lc003 | ETL:LiQ |
| | 100 | 1720 | 100 | 400 | 350 |
| A16 | HTM1:NDP-9 | HTM1 | HTM2 | H1:H2:IrLa005Lb009Lc004 | ETL:LiQ |
| | 100 | 1720 | 100 | 400 | 350 |
| A17 | HTM1:NDP-9 | HTM1 | HTM2 | H1:H2:IrLa005Lb009Lc025 | ETL:LiQ |
| | 100 | 1720 | 100 | 400 | 350 |
| A18 | HTM1:NDP-9 | HTM1 | HTM2 | H1:H2:IrLa005Lb009Lc027 | ETL:LiQ |
| | 100 | 1720 | 100 | 400 | 350 |
| Comparative | HTM1:NDP-9 | HTM1 | HTM2 | H1:H2:Reference compound 1 | ETL:LiQ |
| Example 1 | 100 | 1720 | 100 | 400 | |
| Comparative | HTM1:NDP-9 | HTM1 | HTM2 | H1:H2:Reference compound 2 | 350 |
| Example 2 | 100 | 1720 | 100 | 400 | |
| Comparative | HTM1:NDP-9 | HTM1 | HTM2 | H1:H2:Reference compound 3 | ETL:LiQ |
| Example 3 | 100 | 1720 | 100 | 400 | |
| Comparative | HTM1:NDP-9 | HTM1 | HTM2 | H1:H2:Reference compound 4 | 350 |
| Example 4 | 100 | 1720 | 100 | 400 | |
| Comparative | HTM1:NDP-9 | HTM1 | HTM2 | H1:H2:Reference compound 5 | ETL:LiQ |
| Example 5 | 100 | 1720 | 100 | 400 | |
| Comparative | HTM1:NDP-9 | HTM1 | HTM2 | H1:H2:Reference compound 6 | ETL:LiQ |
| Example 6 | 100 | 1720 | 100 | 400 | |

Evaluation: Properties of devices obtained above were tested. In various examples and comparative examples, a constant-current power supply (Keithley 2400) was used, a current at a fixed density was used for flowing through light-emitting elements, and a spectroradiometer (CS 2000) was used for testing the light-emitting spectrum. Meanwhile, the voltage value was measured, and the time (LT90) when the brightness was reduced to 90% of the initial brightness was tested. Results are as follows. The 10 current efficiency and the device service life are calculated with the value of the reference compound 5 as 100%.

| | Starting | | Chromaticity | |
|---|---|---|---|---|
| | voltage@20 | Current | coordinate | LT90@ |
| | mA/cm² | efficiency | @20 mA/cm2 | 8000 |
| | V | @20 mA/cm2 | CIEx, CIEy | nits |
| Example A1 | 4.34 | 128 | 0.702, 0.298 | 163 |
| Example A2 | 4.37 | 135 | 0.701, 0.299 | 149 |
| Example A3 | 4.34 | 135 | 0.701, 0.299 | 150 |
| Example A4 | 4.35 | 136 | 0.703, 0.297 | 185 |
| Example A5 | 4.32 | 133 | 0.701, 0.298 | 142 |
| Example A6 | 4.31 | 136 | 0.703, 0.296 | 144 |
| Example A7 | 4.33 | 137 | 0.701, 0.299 | 146 |
| Example A8 | 4.36 | 132 | 0.702, 0.298 | 147 |
| Example A9 | 4.32 | 134 | 0.703, 0.296 | 162 |
| Example A10 | 4.34 | 142 | 0.702, 0.298 | 156 |
| Example A11 | 4.35 | 138 | 0.701, 0.299 | 161 |
| Example A12 | 4.32 | 144 | 0.703, 0.297 | 172 |
| Example A13 | 4.36 | 137 | 0.702, 0.297 | 148 |
| Example A14 | 4.36 | 140 | 0.701, 0.298 | 152 |
| Example A15 | 4.34 | 133 | 0.701, 0.299 | 141 |
| Example A16 | 4.33 | 135 | 0.700, 0.299 | 153 |
| Example A17 | 4.35 | 132 | 0.701, 0.298 | 163 |
| Example A18 | 4.34 | 134 | 0.701, 0.298 | 167 |
| Comparative | 5.23 | 75 | 0.700, 0.299 | 51 |
| Example 1 | | | | |
| Comparative | 5.15 | 72 | 0.701, 0.298 | 50 |
| Example 2 | | | | |
| Comparative | 5.34 | 74 | 0.703, 0.296 | 42 |
| Example 3 | | | | |
| Comparative | 5.52 | 63 | 0.702, 0.297 | 37 |
| Example 4 | | | | |
| Comparative | 4.88 | 100 | 0.701, 0.298 | 100 |
| Example 5 | | | | |
| Comparative | 4.74 | 95 | 0.702, 0.298 | 118 |
| Example 6 | | | | |

Through comparison of the data in the above table, it can be seen that compared with reference compounds, the compound of the present invention used as a dopant in an organic electroluminescent device with the same chromaticity coordinate has more excellent properties, such as driving voltage, luminous efficiency, and device service life.

The comparison of emission wavelengths in a dichloromethane solution is defined as follows. A corresponding compound is prepared into a 10-5 mol/L solution with dichloromethane, and the emission wavelength is tested by a Hitachi (HITACH) F2700 fluorescence spectrophotometer to obtain the wavelength at a maximum emission peak. Test results are shown as follows.

| Material | PL peak wavelength/nm |
|---|---|
| Ir(La002)₂ Lb005 | 631 |
| Ir(La005)₂ Lb005 | 628 |
| Ir(La018)₂ Lb005 | 632 |
| Ir(La025)₂ Lb005 | 632 |
| Ir(La031)₂ Lb005 | 630 |
| Ir(La032)₂ Lb005 | 631 |
| Ir(La033)₂ Lb005 | 631 |
| Ir(La042)₂ Lb005 | 629 |
| Ir(La050)₂ Lb005 | 631 |
| Ir(La068)₂ Lb005 | 631 |
| Ir(La079)₂ Lb005 | 630 |
| Ir(La086)₂ Lb005 | 631 |
| Ir(La005)₂ Lb009 | 631 |
| Ir(La005)₂ Lb018 | 632 |
| Ir La005 Lb009 Lc003 | 630 |
| Ir La005 Lb009 Lc004 | 629 |
| Ir La005 Lb009 Lc025 | 631 |
| Ir La005 Lb009 Lc027 | 631 |
| Reference compound 1 | 610 |
| Reference compound 2 | 637 |
| Reference compound 3 | 611 |
| Reference compound 4 | 608 |
| Reference compound 5 | 616 |

Through comparison of the data in the above table, it can be seen that compared with reference compounds, the metallic iridium compound of the present invention has a larger red shift, which can meet industrial demands for dark red light, especially for a color gamut of BT2020.

Comparison of the sublimation temperature is as follows. The sublimation temperature is defined as the temperature corresponding to an evaporation rate of 1 Å/s at a vacuum degree of 10⁻⁷ Torr. Test results are shown as follows.

| Material | Sublimation temperature |
|---|---|
| Ir(La002)₂ Lb005 | 271 |
| Ir(La018)₂ Lb005 | 273 |
| Ir(La033)₂ Lb005 | 273 |
| Ir(La068)₂ Lb005 | 270 |
| Ir(La079)₂ Lb005 | 265 |
| Ir(La086)₂ Lb005 | 266 |
| Ir La005 Lb009 Lc003 | 272 |
| Reference compound 1 | 280 |
| Reference compound 2 | 288 |
| Reference compound 3 | 286 |
| Reference compound 4 | 276 |
| Reference compound 5 | 268 |

Through comparison of the data in the above table, it can be seen that the metallic iridium compound of the present invention has low sublimation temperature, which is conducive to industrial application.

Compared with the prior art, the present invention unexpectedly provides better device luminous efficiency, improved service life, lower sublimation temperature and more saturated red luminescence through special collocation of substituents. The above results show that the compound of the present invention has the advantages of low sublimation temperature, high optical and electrochemical stability, high color saturation, high luminous efficiency, long device service life and the like, and can be used in an organic electroluminescent device. In particular, the compound has the possibility of being applied to the OLED industry as a red light-emitting dopant, especially for display, illumination and vehicle taillights.

1. An organometallic iridium compound, having a general formula of Ir(La)(Lb)(Lc), wherein La is a structure as shown in a formula (1): 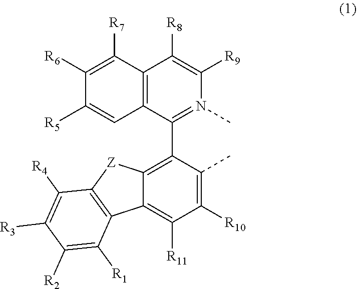
wherein a dotted line refers to a position connected to the metal Ir;
Z is O, S or Se;
R₁-R₁₁ are independently selected from hydrogen, deuterium, halogen, cyano, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₃-C₂₀heterocycloalkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl, or two adjacent groups of R₁-R₄ are connected to each other to form an aliphatic ring;
R₁₀ is not hydrogen, deuterium, halogen or cyano;
at least one of R₅-R₇ is substituted or unsubstituted C₆-C₃₀ aryl, or substituted or unsubstituted C₂-C₃₀ heteroaryl;
the heteroalkyl, the heterocycloalkyl and the heteroaryl at least contain one O, N, or S heteroatom;
and the “substituted” refers to substitution with deuterium, F, Cl, Br, C₁-C₆ alkyl, C₃-C₆ cycloalkyl, C₁-C₆ alkyl substituted amino, cyano, isocyano, or phosphino, and the substitution ranges from a single substitution number to a maximum substitution number;
Lb is a structure as shown in a formula (2): 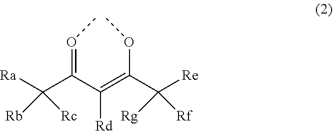
where a dotted line refers to a position connected to the metal Ir;
Ra-Rg are independently selected from hydrogen, deuterium, halogen, substituted or unsubstituted C₁-C₁₀ alkyl, substituted or unsubstituted C₃-C₂₀ cycloalkyl, substituted or unsubstituted C₁-C₁₀heteroalkyl, and substituted or unsubstituted C₃-C₂₀heterocycloalkyl, or any two of Ra, Rb and Rc are connected to form an aliphatic ring, and any two of Re, Rf and Rg are connected to form an aliphatic ring;
the heteroalkyl and the heterocycloalkyl at least contain one O, N, or S heteroatom;
and the “substituted” refers to substitution with deuterium, F, Cl, Br, C₁-C₄ alkyl, C₁-C₄ alkoxyl, C₃-C₆cycloalkyl, C₁-C₄
alkyl substituted amino, cyano,, isocyano, or phosphino;
Lc is a monoanionic bidentate ligand, and the Lc is different from the Lb and is not an OO ligand;
the Lc and the La are the same or different, and the different indicates different parent nuclear structures, a same parent nuclear structure with different substituents, or a same parent nuclear structure with same substituents at different substituent positions;
and any two or three of the La, the Lb and the Lc are connected to each other to form a polydentate ligand.

2. The organometallic iridium compound according to claim 1, wherein the R₆ is substituted or unsubstituted C₆-C₃₀ alkyl, or substituted or unsubstituted C₂-C₃₀ heteroaryl.

3. The organometallic iridium compound according to claim 2, wherein the R₆ is substituted or unsubstituted C₆-C₁₈ alkyl, or substituted or unsubstituted C₂-C₁₇ heteroaryl.

4. The organometallic iridium compound according to claim 2, wherein the R₁₀ is substituted or unsubstituted C₁-C₆ alkyl, or substituted or unsubstituted C₃-C₆ cycloalkyl, and the “substituted” refers to substitution with deuterium, F, C₁-C₅ alkyl, or C₃-C₆ cycloalkyl.

5. The organometallic iridium compound according to claim 2, wherein at least one of the R₈ and the R₉ is not hydrogen, deuterium, halogen or cyano.

6. The organometallic iridium compound according to claim 5, wherein at least one of the R₈ and the R₉ is substituted or unsubstituted C₁-C₆ alkyl, or substituted or unsubstituted C₃-C₆ cycloalkyl.

7. The organometallic iridium compound according to claim 2, wherein the R₁-R₄ are hydrogen.

8. The organometallic iridium compound according to claim 2, wherein the Z is O.

9. The organometallic iridium compound according to claim 2, wherein the Lc and the La are different.

10. The organometallic iridium compound according to claim 9, wherein the Lc is a structure as shown in a formula (3): 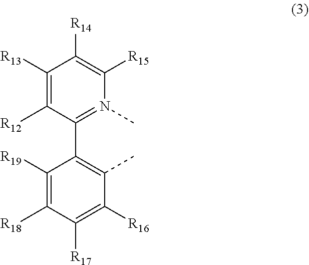
wherein a dotted line refers to a position connected to the metal Ir;
R₁₂-R₁₉ are independently selected from hydrogen, deuterium, halogen, cyano, hydroxyl, amino, amino group, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₂-C₁₀ alkenyl, substituted or unsubstituted C₂-C₁₀ alkynyl, substituted or unsubstituted C₆-C₁₈ aryl, substituted or unsubstituted C₂-C₁₇ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl;
at least two of the R₁₆-R₁₉ are not hydrogen;
and at least one group pf two adjacent groups of the R₁₂-R₁₅ may form an aromatic ring as shown in a formula (4) below: 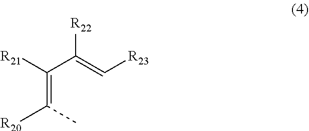
wherein in the formula (4),
a dotted line refers to a position connected to a pyridine ring;
R₂₀-R₂₃ are independently selected from hydrogen, deuterium, halogen, cyano, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₂-C₁₀ alkenyl, substituted or unsubstituted C₂-C₁₀ alkynyl, substituted or unsubstituted C₆-C₁₈ aryl, substituted or unsubstituted C₂-C₁₇ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl, or two adjacent groups of the R₂₀-R₂₃ are connected to each other to form an aliphatic ring or an aromatic ring;
the heteroalkyl and the heteroaryl at least contain one O, N, or S heteroatom;
and the “substituted” refers to substitution with deuterium, F, Cl, Br, C₁-C₆alkyl, C₃-C₆ cycloalkyl, C₁-C₆ alkyl substituted amino, cyano, isocyano, or phosphino, and the substitution ranges from a single substitution number to a maximum substitution number.

11. The organometallic iridium compound according to claim 10, wherein the Lc has one of the following structural formulas, or is partially or completely deuterated or fluorinated correspondingly,

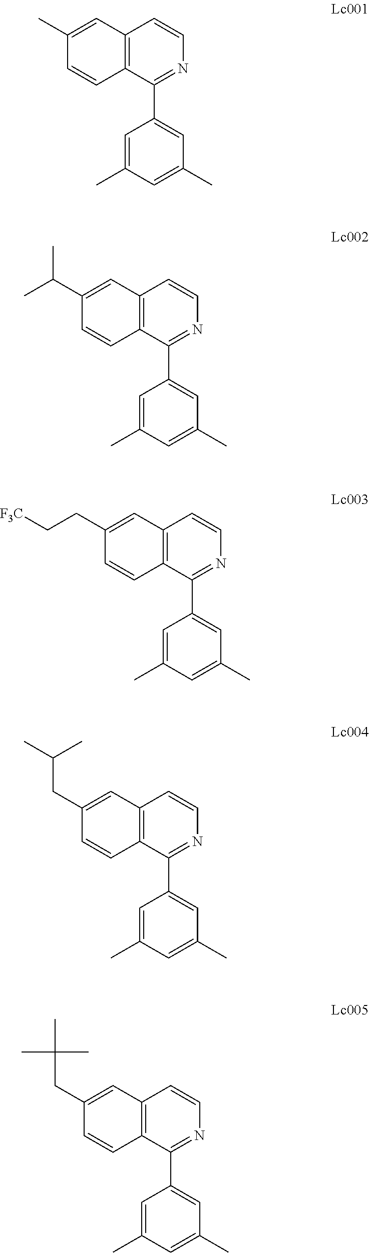

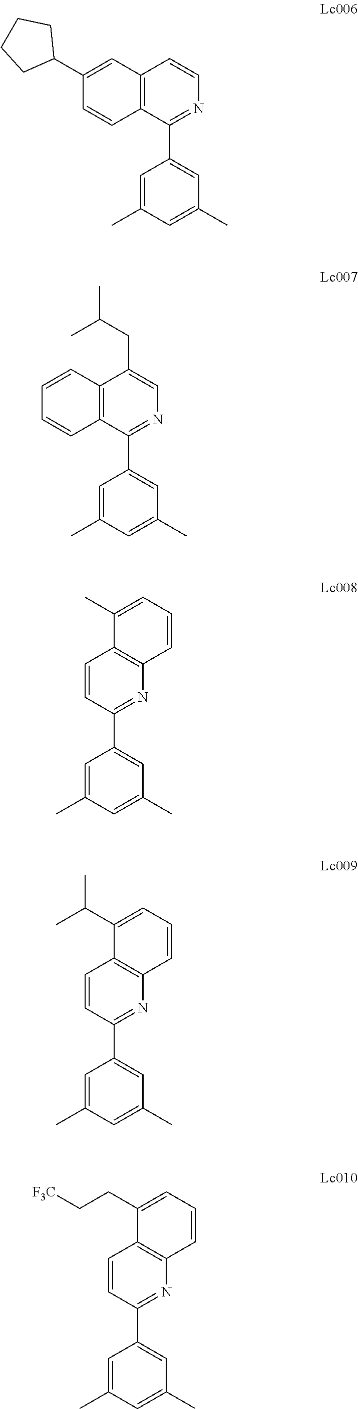

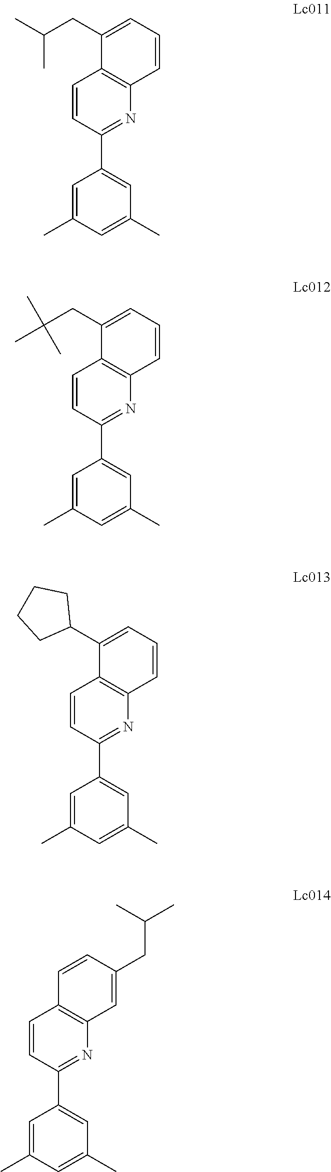 [SMILES: Cc1cc(C)cc(-c2ccc3c(CC(C)C)cccc3n2)c1 ; Cc1cc(C)cc(-c2ccc3c(CC(C)(C)C)cccc3n2)c1 ; Cc1cc(C)cc(-c2ccc3c(C4CCCC4)cccc3n2)c1 ; Cc1cc(C)cc(-c2ccc3ccc(CC(C)C)cc3n2)c1]

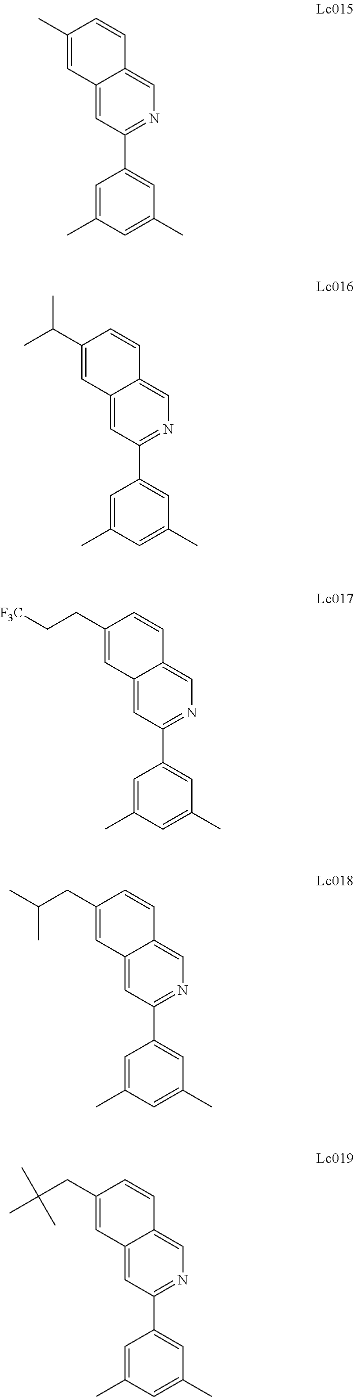

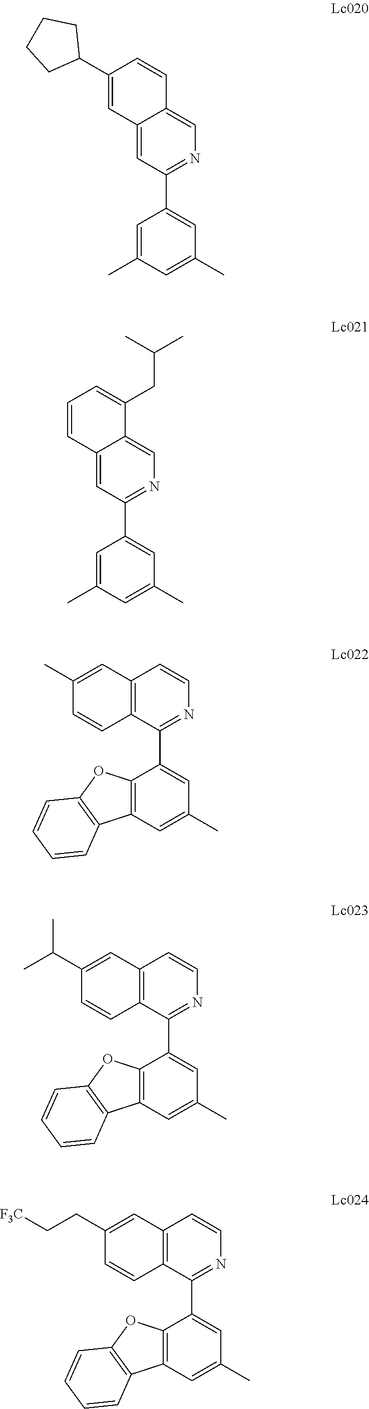

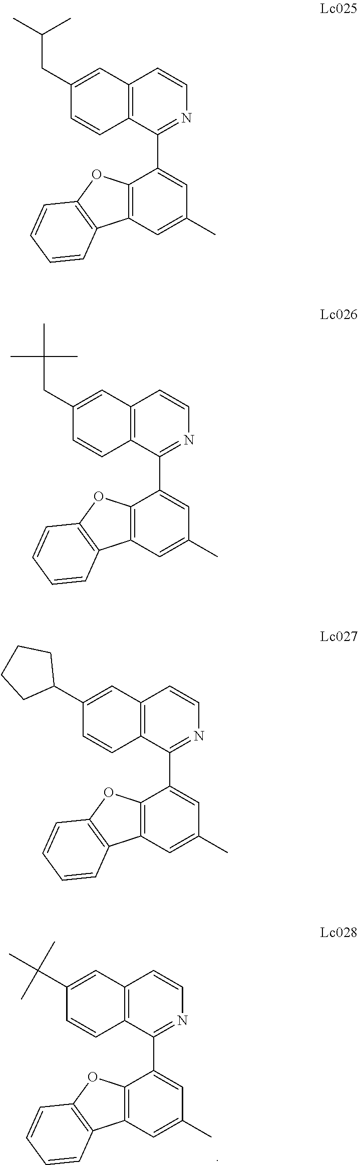

12. The organometallic iridium compound according to claim 2, wherein the La has one of the following structural formulas, or is partially or completely deuterated or fluorinated correspondingly,

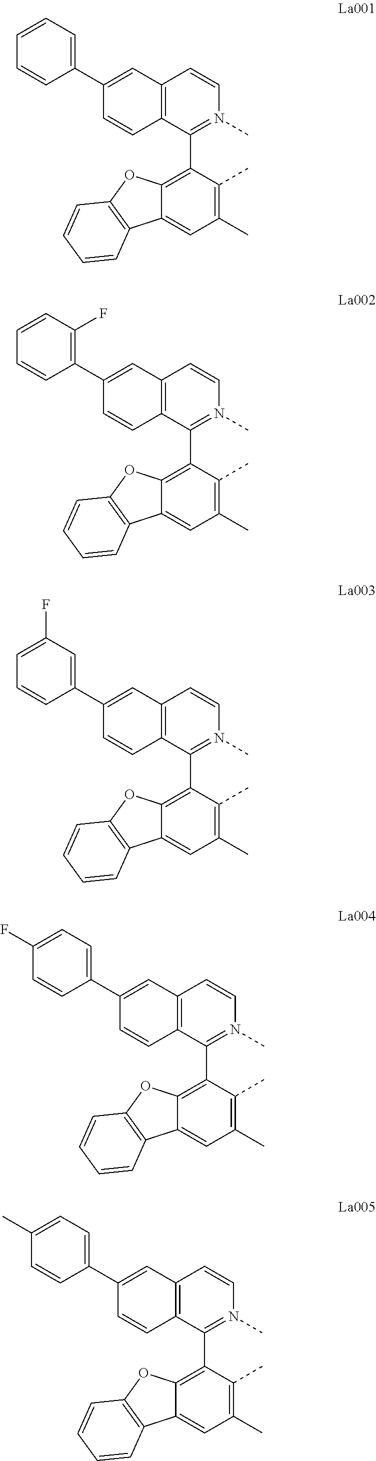

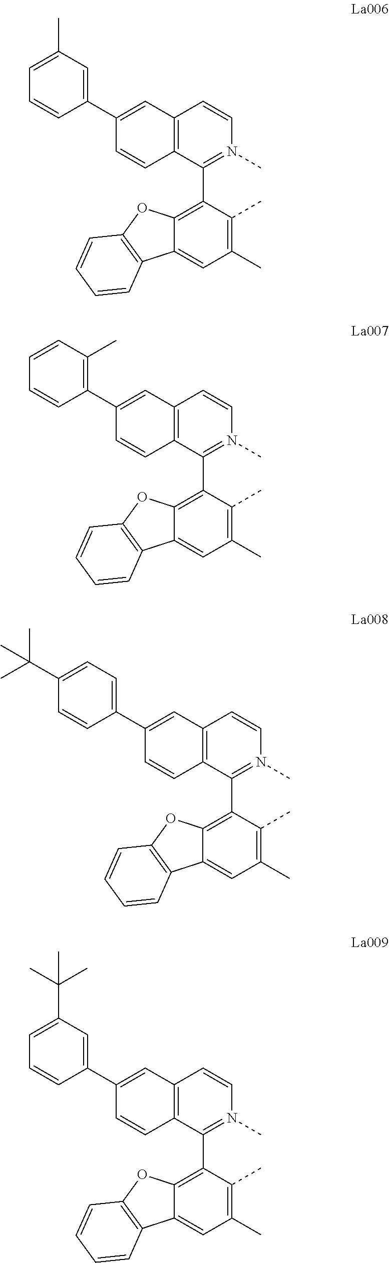

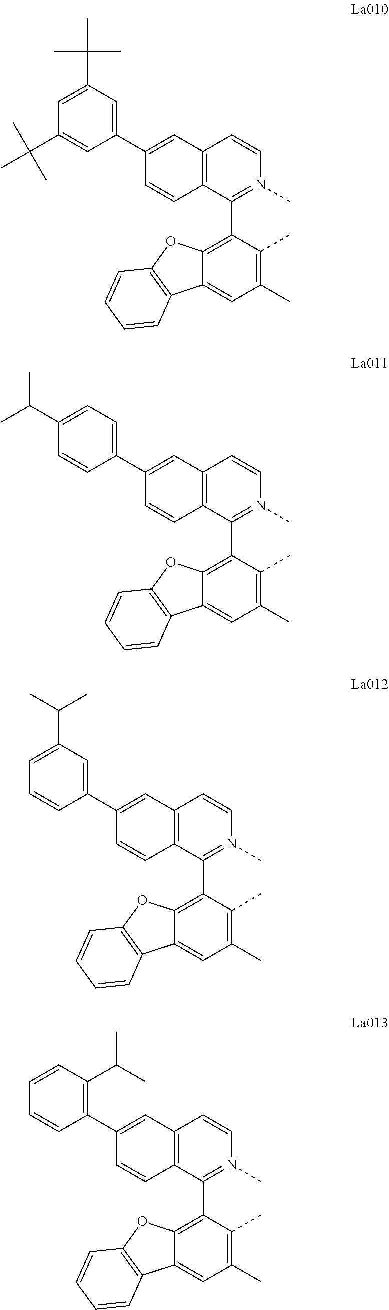

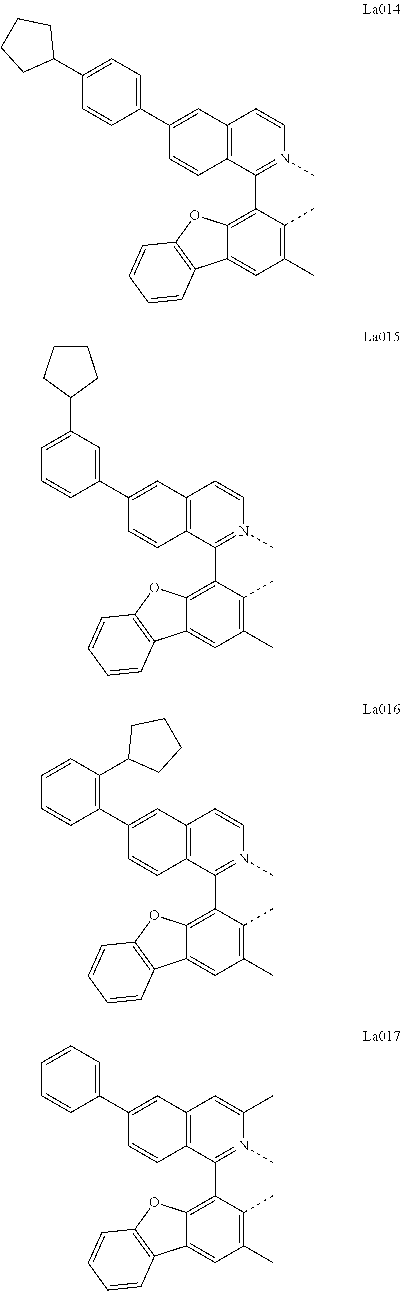

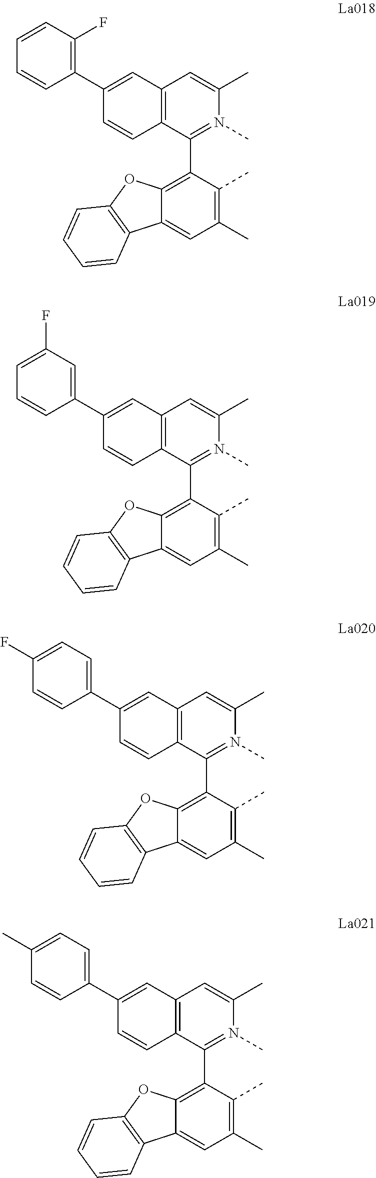

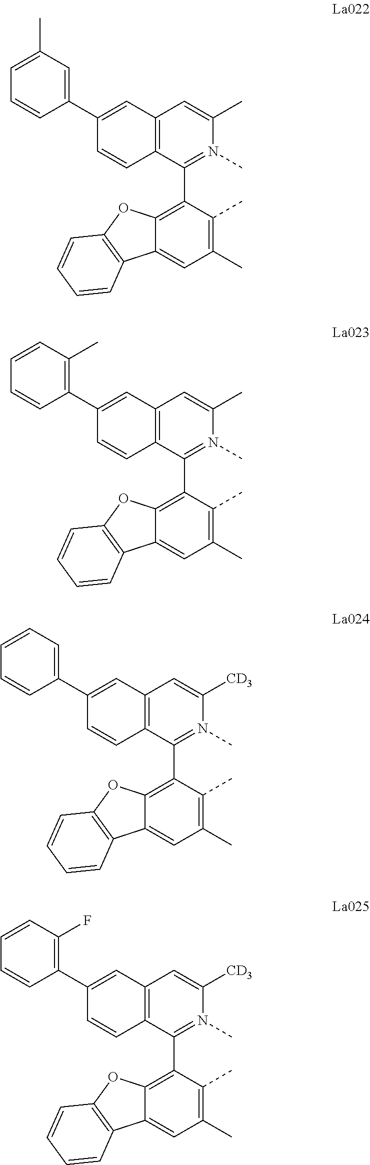

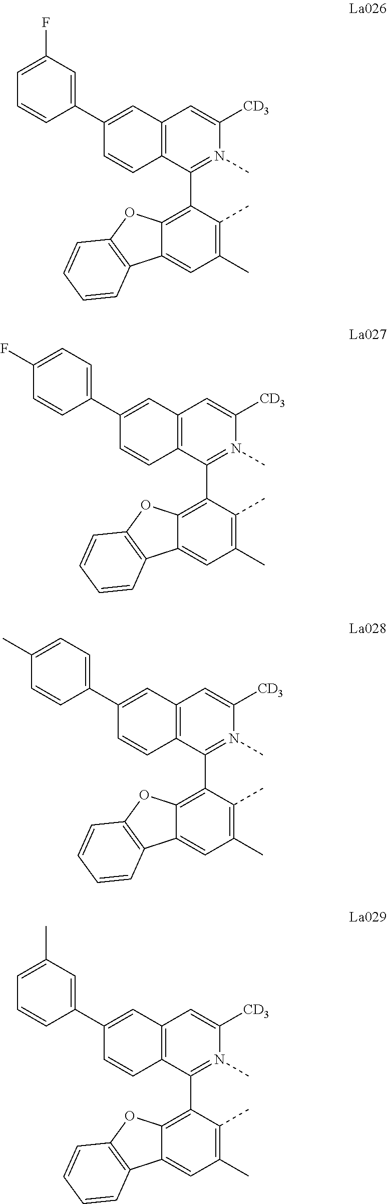

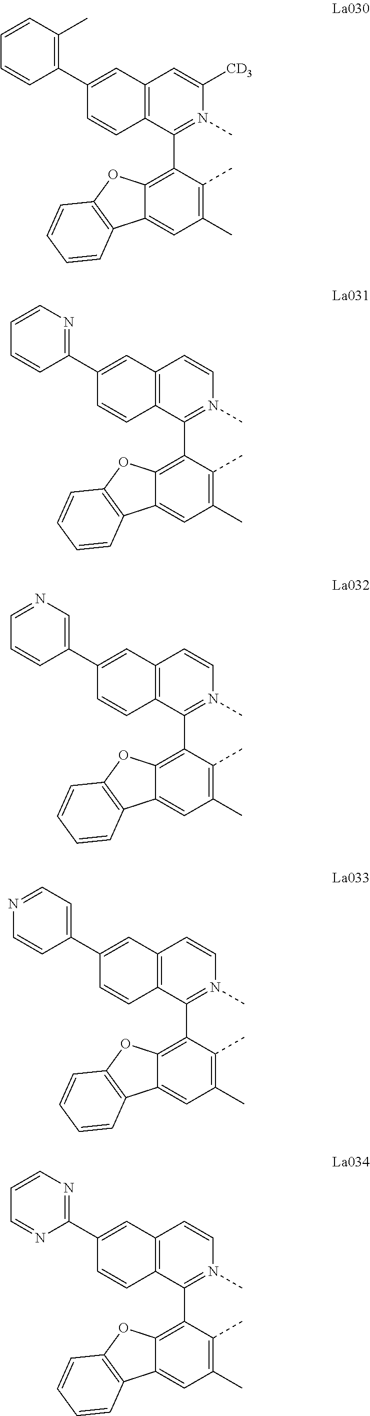

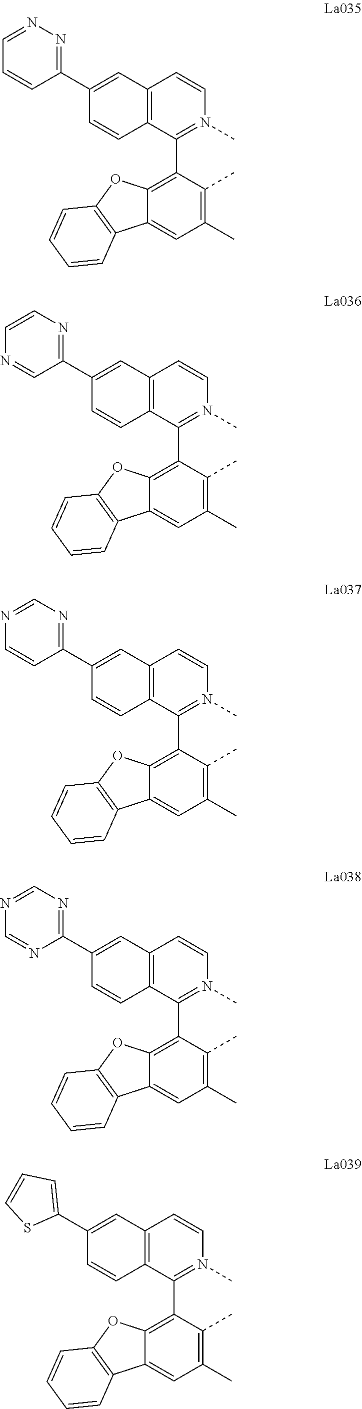

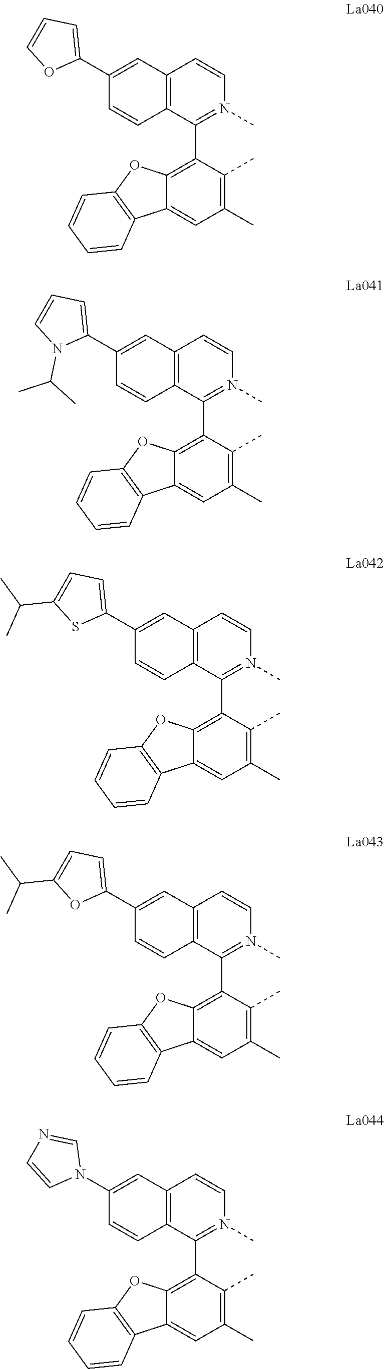

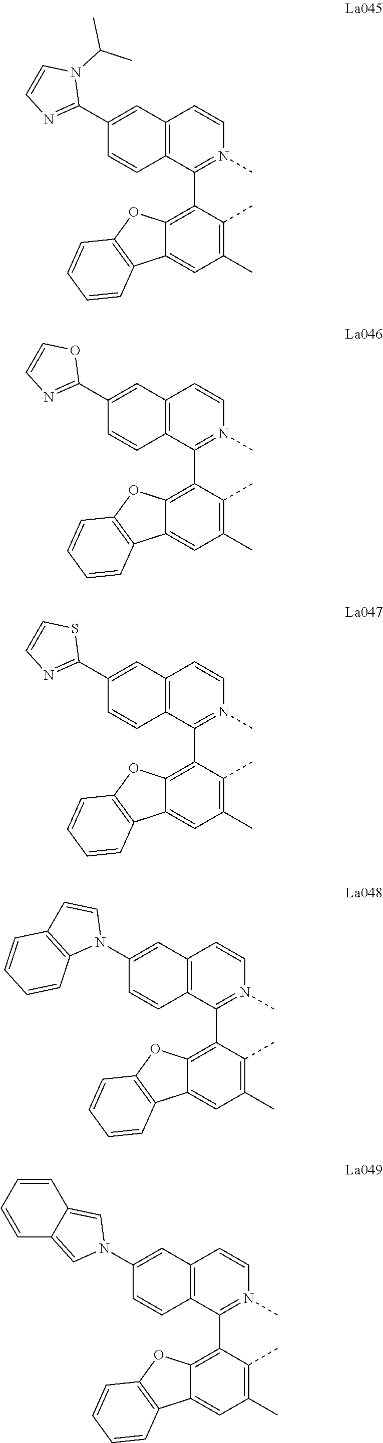

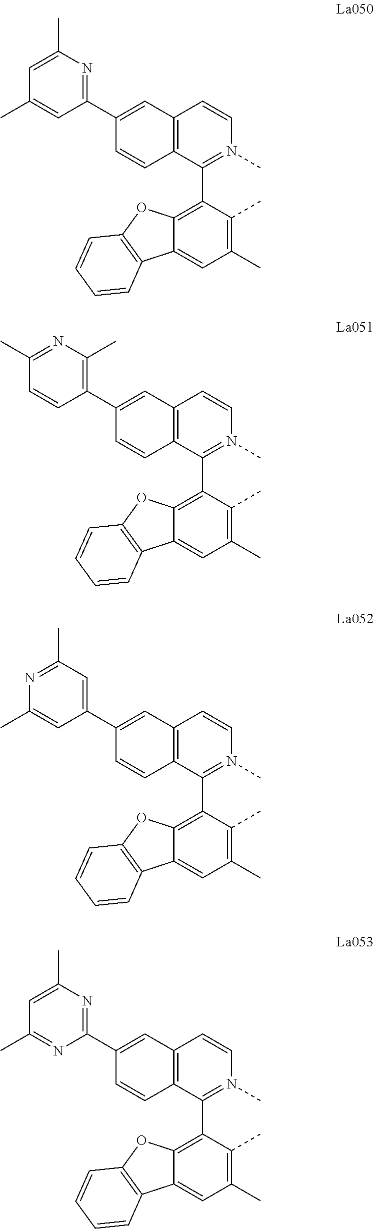

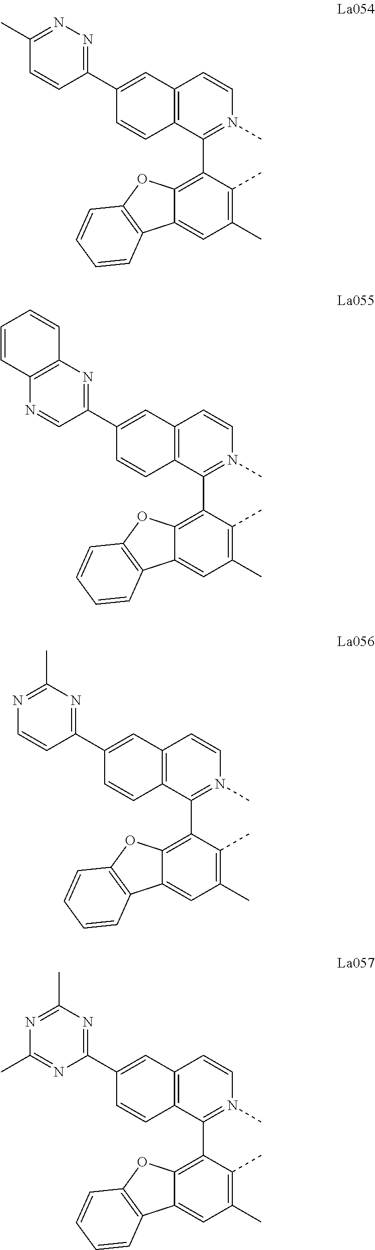

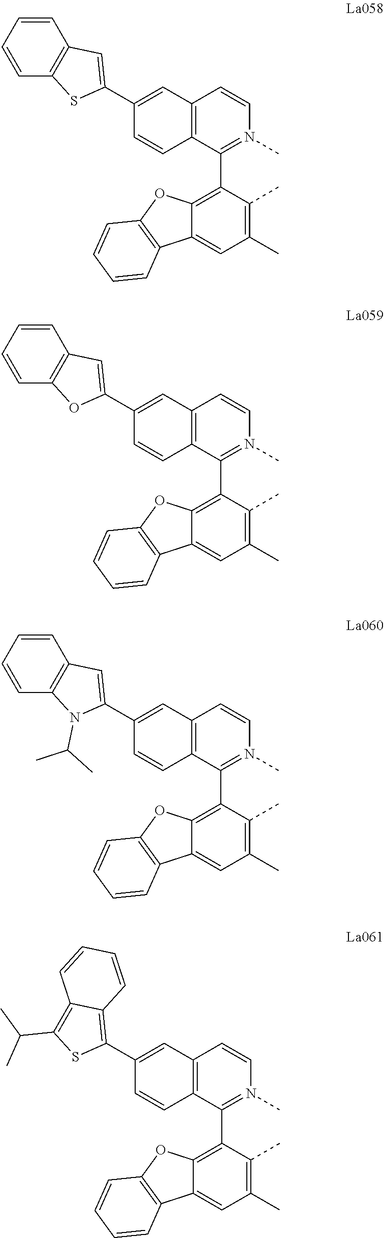

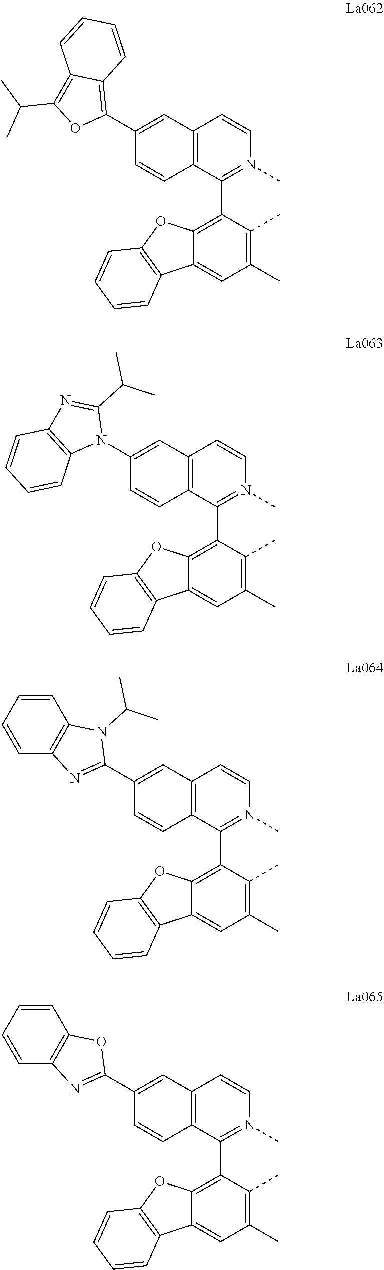

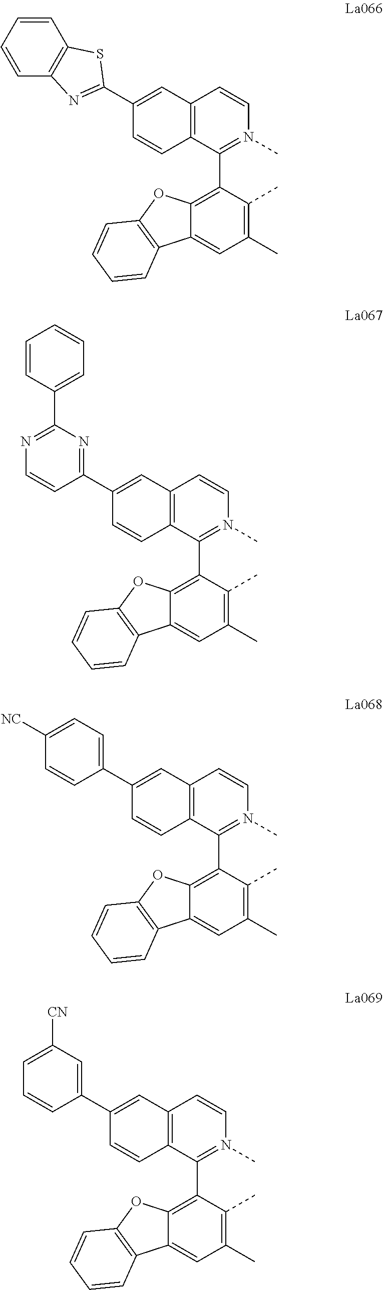

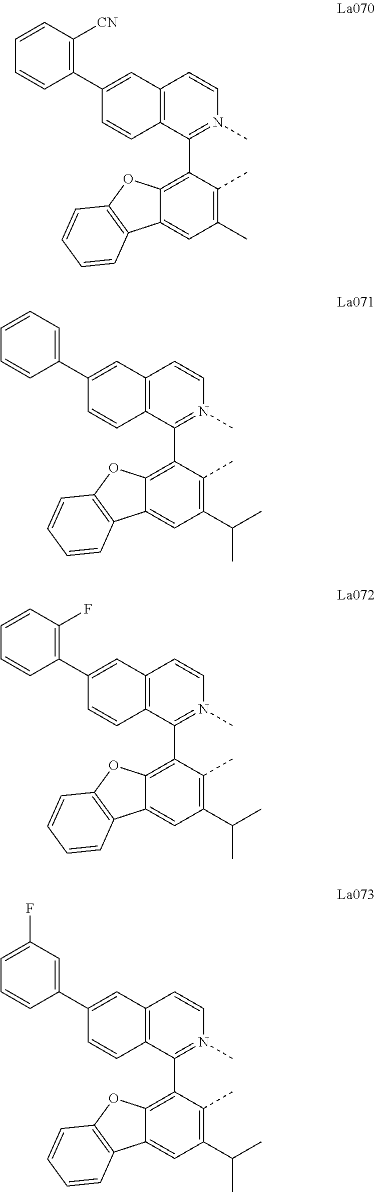

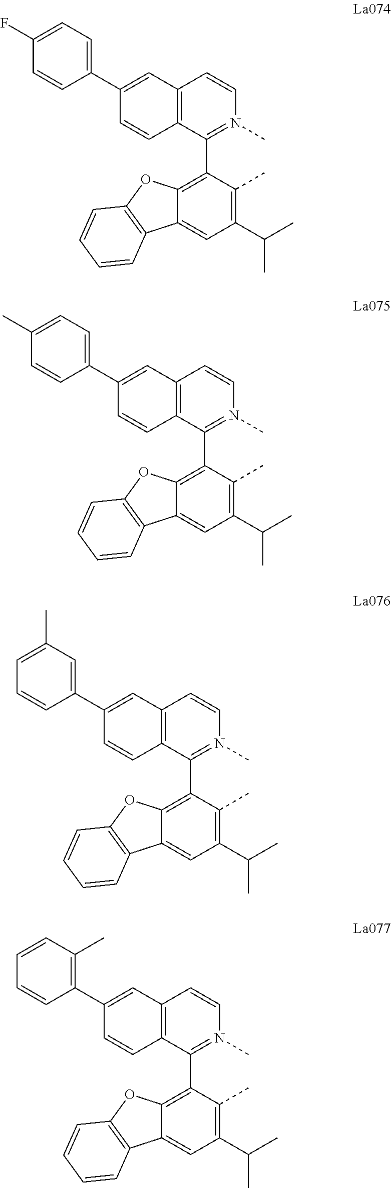

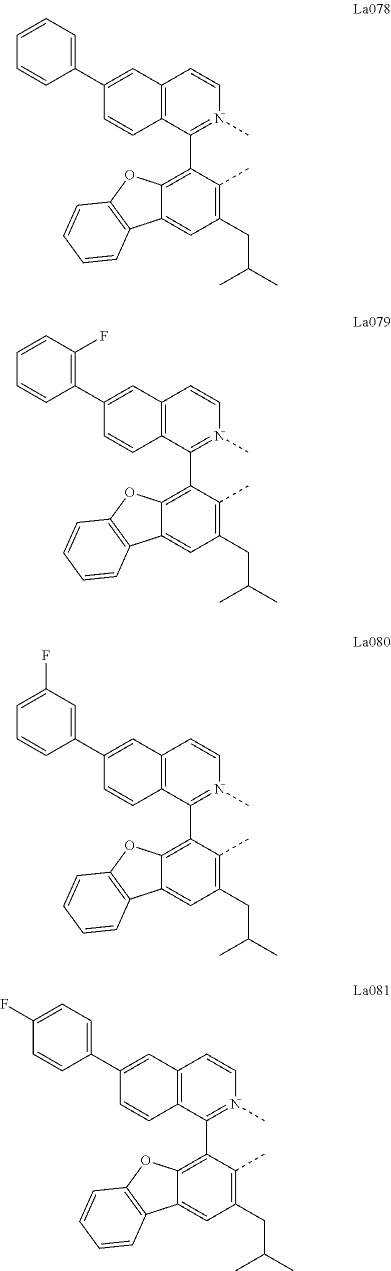

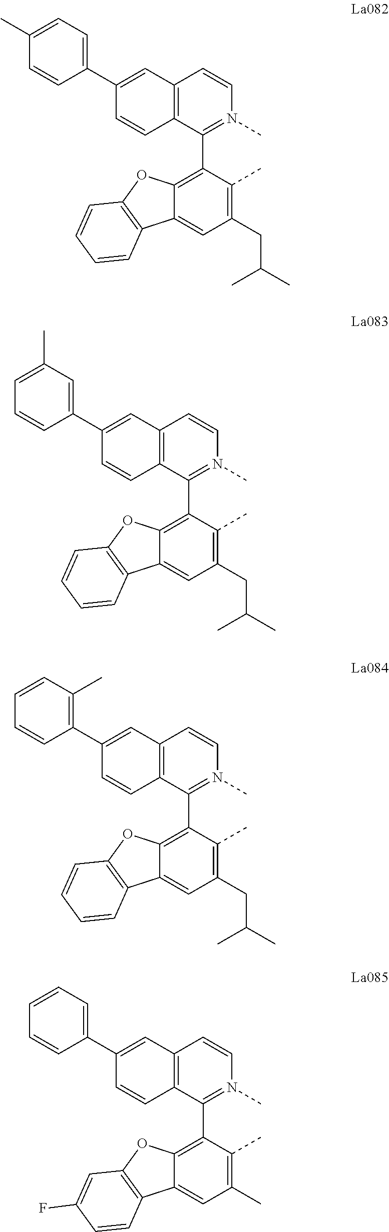

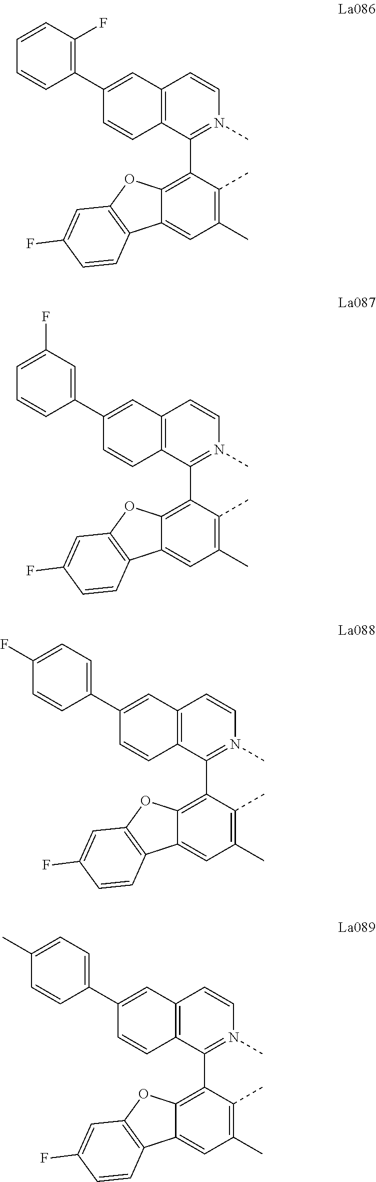

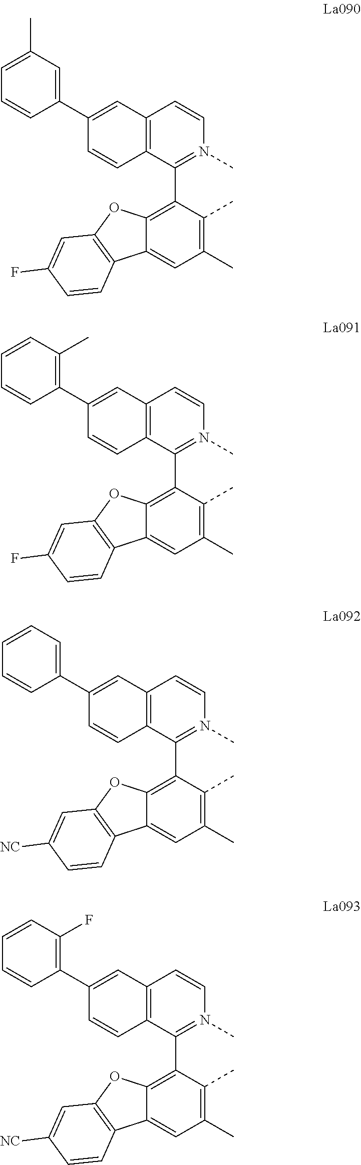

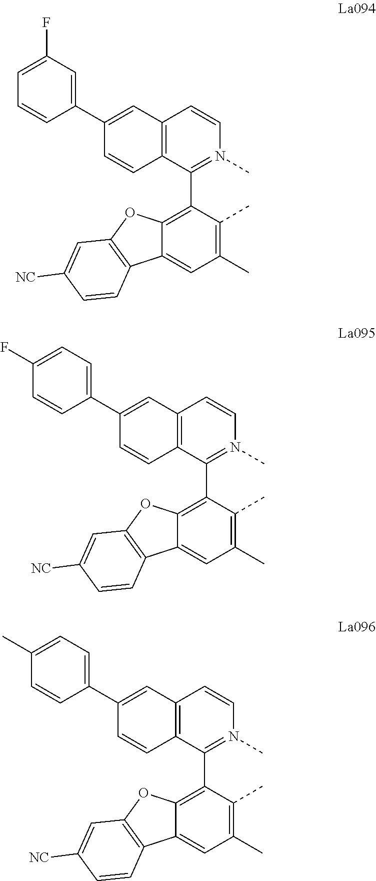

13. The organometallic iridium compound according to claim 2, wherein the Lb has one of the following structural formulas, or is partially or completely deuterated or fluorinated correspondingly,

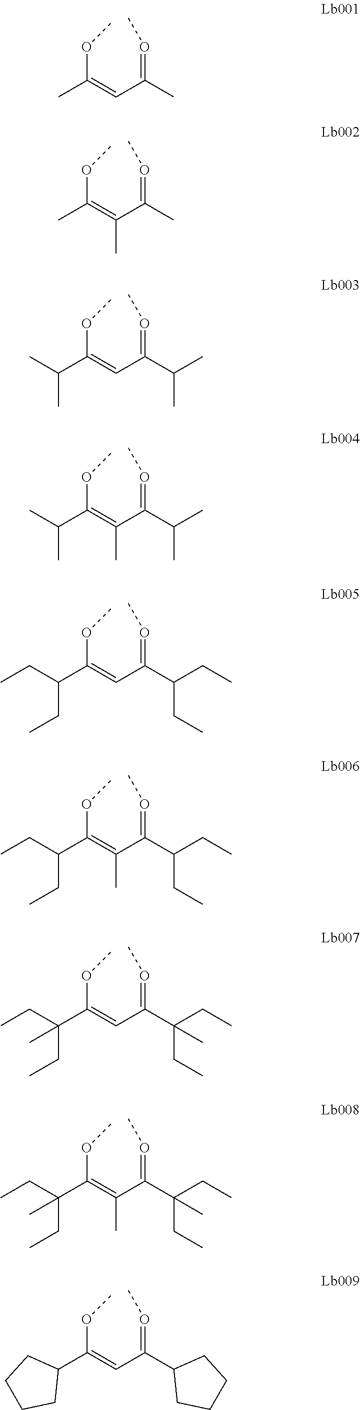

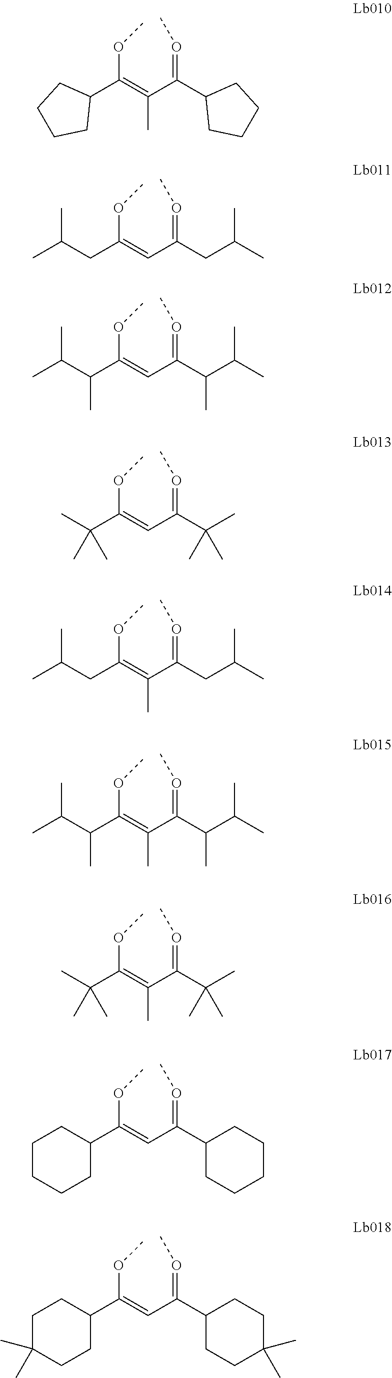

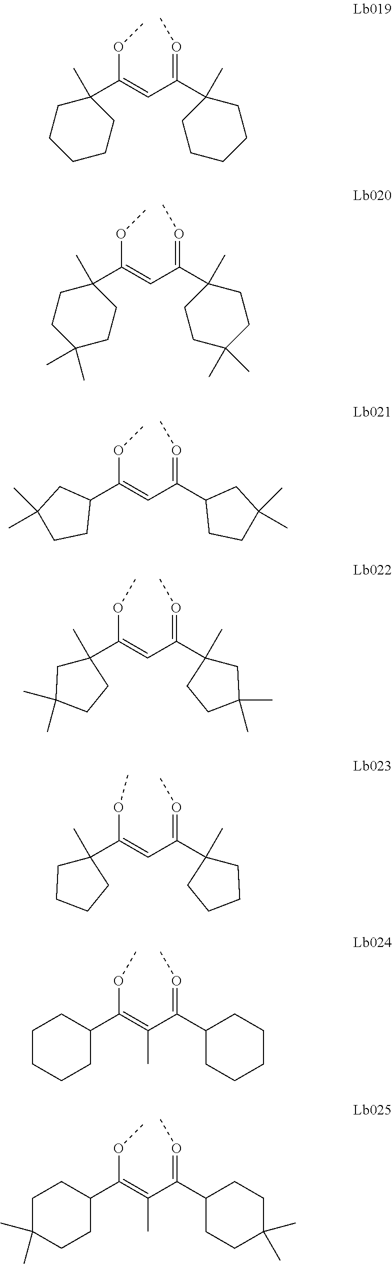

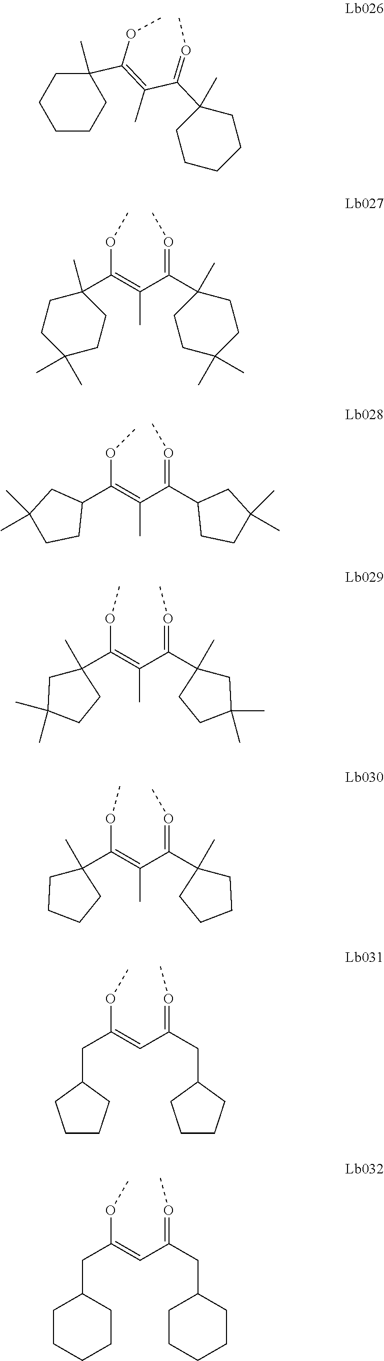

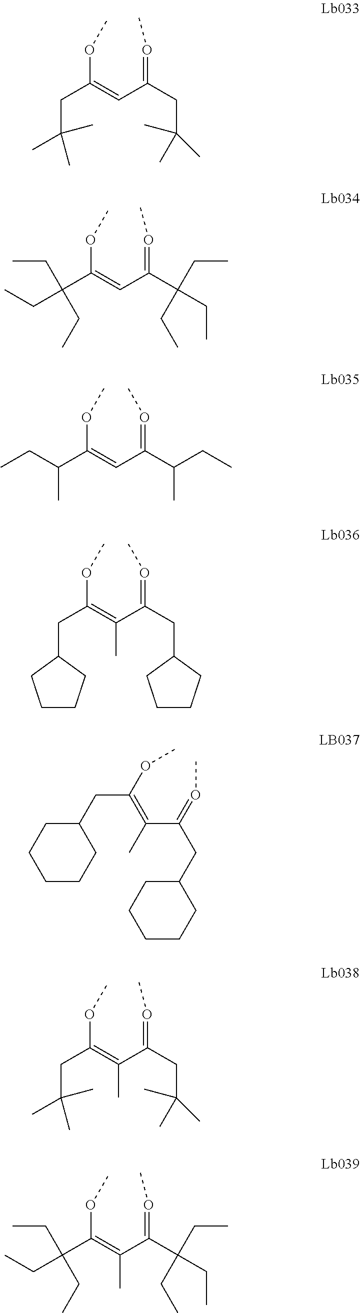

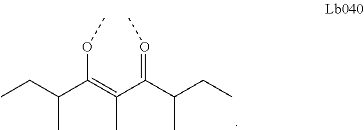

14. An electroluminescent device, comprising a cathode, an anode and an organic layer arranged between the cathode and the anode, wherein the organic layer comprises the organometallic iridium compound according claim 1.

15. The electroluminescent device according to claim 14, wherein the organic layer comprises a light-emitting layer, and the organometallic iridium compound according to claim 1 is used a red light-emitting doping material for the light-emitting layer.

16. A ligand La, having a structural formula below: 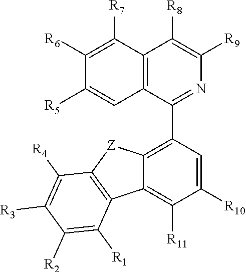
wherein R₁-R₁₁ and Z are defined according to claim 8.

17. The electroluminescent device according to claim 1, wherein the organic layer comprises a hole injection layer, and the organometallic iridium compound according to any one of claims 1 is used as a hole injection material in the hole injection layer.
